(19) United States
(12) Patent Application Publication
(10) Pub. No.: US 2019/0339204 A1
(43) Pub. Date: Nov. 7, 2019

(54) CO-LOCALIZATION AT MOLECULAR RESOLUTION OF MULTIPLE FLUORESCENCE CHANNELS ACQUIRED USING OPTICAL MICROSCOPY

(71) Applicant: ALBERT EINSTEIN COLLEGE OF MEDICINE, Bronx, NY (US)

(72) Inventors: Robert H. Singer, New York, NY (US); Carolina Eliscovich, Bronx, NY (US); Shailesh M. Shenoy, New Rochelle, NY (US)

(73) Assignee: ALBERT EINSTEIN COLLEGE OF MEDICINE, Bronx, NY (US)

(21) Appl. No.: 16/476,582

(22) PCT Filed: Jan. 19, 2018
(86) PCT No.: PCT/US18/14313

§ 371 Date: Jul. 9, 2019

Related U.S. Application Data
(60) Provisional application No. 62/451,096, filed on Jan. 27, 2017.

(51) Int. Cl. G01N 21/64; G02B 21/02; G02B 21/16

(52) U.S. Cl. CPC G01N 21/6458; G01N 33/582; G02B 21/16; G02B 21/02

(57) ABSTRACT

A method for improving the performance of a fluorescence microscopy imaging system and for correcting chromatic aberration of an optical objective in a fluorescence microscopy system.

### CROSS-REFERENCE TO RELATED APPLICATION

This application claims benefit of U.S. Provisional Application No. 62/451,096, filed Jan. 27, 2017, the contents of which are hereby incorporated by reference.

### STATEMENT OF GOVERNMENT SUPPORT

This invention was made with government support under grant number NS083085 awarded by the National Institutes of Health. The government has certain rights in the invention.

### BACKGROUND OF THE INVENTION

Throughout this application various publications are referred to. Full citations for the references may be found at the end of the specification. The disclosures of these publications are hereby incorporated by reference in their entirety into the subject application to more fully describe the art to which the subject invention pertains.

RNA-binding proteins (RBPs) specifically recognize and bind with RNA regulating its life cycle (1, 2). Dysfunctional RNA-protein interaction represents one of causes of genetic disorders that vary from neurodevelopmental and neurodegenerative diseases to cancer (3-9). Traditionally, RNA-protein interactions have been investigated by ensemble biochemistry approaches including affinity purification and crosslinking and immunoprecipitation-based techniques (reviewed in (10, 11)). However, these methods may report adventitious RNA-protein associations that would occur after lysis of cells (12, 13), or functionally important complexes may not survive the procedure. Importantly, ensemble biochemistry studies lack morphological information, particularly essential for neurons.

Currently there is no method to verify whether these biochemical techniques determine real interactions that take place in the cell. Standard wide-field microscopy has been utilized to reveal interactions by “colocalizing” two fluorescent tags. Technically, colocalization refers to two or more fluorescent molecules emitting different wavelengths of light that superimpose within an indeterminate microscopic resolution. Biologically, colocalization implies the association between these molecules. However, their physical association occurs at a dimension not usually achievable by light microscopy, since it occurs below the diffraction limit (approximately 250 nm). Thus as currently practiced, “colocalization” is a suggestion of spatial correlation but does not rule out random association.

The present invention addresses the need to correct chromatic aberration in optical fluorescence microscopy.

### SUMMARY OF THE INVENTION

This work represents a solution to the historical problem of registration of two colors in optical fluorescence systems, achieved here in molecular resolution (10 nm). The invention provides, inter alia, a method to correct the intrinsic aberration of the commercial microscope objectives, each of which is unique. This allows the use of imaging to characterize the interaction of two molecules while in their native environment. This method has been applied in the study of the interaction of mRNAs with putative RNA binding proteins isolated by standard techniques to verify which bind and which do not using a combined approach to detect both RNA and proteins. The results surprisingly indicate that some proteins thought to bind mRNAs in fact do not when analyzed by this high resolution imaging technique.

A method is provided for improving the performance of a fluorescence microscopy imaging system comprising an optical objective lens, a field of view, an imaging detector, and at least a first and a second fluorescent molecule, each of which fluoresces at a different wavelength than the other and each of which has a different excitation radiation peak than the other fluorescent molecule, the method comprising:
providing in a field of view of the fluorescence microscopy system a plurality of fluorescent beads capable of fluorescing at each of the different wavelengths of the first and second fluorescent molecules, wherein the beads have a diameter lower than a diffraction limit of the optical fluorescence microscopy system;
irradiating the plurality of fluorescent beads at an excitation radiation peak of the first fluorescent molecule and sequentially imaging the fluorescence of each of the plurality of beads within field of view of the fluorescence microscopy system and at a plurality of different z-dimension positions;
irradiating the plurality of fluorescent beads at an excitation radiation peak of the second fluorescent molecule and sequentially imaging the fluorescence of each of the plurality of beads within field of view of the fluorescence microscopy system and at a plurality of different z-dimension positions;
locating, from a point spread function of the fluorescence of each bead imaged at the excitation radiation peak of the first fluorescent molecule, the x,y coordinates of a centroid for each bead at each z-dimension position;
locating, from a point spread function of the fluorescence of each bead imaged at the excitation radiation peak of the second fluorescent molecule, the x,y coordinates of a centroid for each bead at each z-dimension position;
calculating, from a difference in the centroid x,y coordinates for each bead at the first and second excitation radiation peaks, a displacement vector for each x,y coordinate in the field of view at each z-dimension position, so as to thereby determine a displacement vector map for the optical objective of the fluorescence microscopy system;
applying the displacement vector map to imaging data obtained for the first and second fluorescent molecule so as to generate a fluorescence data image corrected for chromatic aberration in the optical objective of the fluorescence microscopy system.

Also provided is a method of correcting for chromatic aberration in a fluorescence microscopy system comprising an optical objective lens, a field of view, an imaging detector, and at least a first and a second fluorescent molecule, each of which fluoresces at a different wavelength than the other and each of which has a different excitation radiation peak than the other fluorescent molecule, the method comprising:
providing in a field of view of the fluorescence microscopy system a plurality of fluorescent beads capable of fluorescing at each of the different wavelengths of the first and second fluorescent molecules, wherein the beads have a diameter lower than a diffraction limit of the optical fluorescence microscopy system;
irradiating the plurality of fluorescent beads at an excitation radiation peak of the first fluorescent molecule and sequentially imaging the fluorescence of each of the plurality of beads within field of view of the fluorescence microscopy system and at a plurality of different z-dimension positions;
irradiating the plurality of fluorescent beads at an excitation radiation peak of the second fluorescent molecule and sequentially imaging the fluorescence of each of the plurality of beads within field of view of the fluorescence microscopy system and at a plurality of different z-dimension positions;
locating, from a point spread function of the fluorescence of each bead imaged at the excitation radiation peak of the first fluorescent molecule, the x,y coordinates of a centroid for each bead at each z-dimension position;
locating, from a point spread function of the fluorescence of each bead imaged at the excitation radiation peak of the second fluorescent molecule, the x,y coordinates of a centroid for each bead at each z-dimension position;
calculating, from a difference in the centroid x,y coordinates for each bead at the first and second excitation radiation peaks, a displacement vector for each x,y coordinate in the field of view at each z-dimension position, so as to thereby determine a displacement vector map for the optical objective of the fluorescence microscopy system;
applying the displacement vector map to imaging data obtained for the first and second fluorescent molecule so as to generate a fluorescence data image corrected for chromatic aberration.

A kit is provided comprising a plurality of broad spectrum fluorescent beads and a non-transitory computer readable medium having instructions thereon for performing the methods described herein in a fluorescence microscopy imaging system.

Also provided is a method of detecting at least two co-localized fluorescent markers, wherein each of the two markers has a different emission spectrum, in a field of view of a fluorescence microscopy imaging system, the method comprising
subjecting an in vitro or in vivo system which has been preloaded with the two markers, wherein at least a portion of the in vitro or in vivo system is within the field of view of the fluorescence microscopy imaging system to irradiation at an excitation spectrum peak of each of the two different markers;
obtaining a fluorescence image for each two markers, when subjected to irradiation, with an optical objective of the fluorescence microscopy imaging system;
correcting the fluorescence images obtained for chromatic aberration of the optical objective at each of the different emission spectrums of the two fluorescent markers by the methods described herein;
determining if the chromatic aberration-corrected fluorescence images show two colocalized different fluorescent markers, so as to thereby detect at least two co-localized fluorescent markers.

Provided is a non-transitory computer-readable medium coupled to the one or more data processing apparatus coupled to a optical microscope fluorescence imaging system, the medium having instructions stored thereon which, when executed by the one or more data processing apparatus, cause the one or more data processing apparatus to perform a method as described hereinabove.

Also provided is a system for improving the performance of a fluorescence microscopy imaging system, comprising:
one or more data processing apparatus;
a graphical user interface; and
a non-transitory computer-readable medium coupled to the one or more data processing apparatus having instructions stored thereon which, when executed by the one or more data processing apparatus, and coupled to an optical microscope fluorescence imaging system, cause the one or more data processing apparatus to perform a method as described hereinabove.

Additional objects of the invention will be apparent from the description which follows.

### BRIEF DESCRIPTION OF THE DRAWINGS

FIG. 1A-1E. Super-registration procedure for dual-color localization microscopy. (A) Registration. A poly-L-lysine coated surface was sparsely loaded with 100 nm diameter fluorescent beads and z-stacks were acquired in Cy5 (green) and Cy3 (red) channels with a wide-field microscope. (B) Chromatic aberration correction. Localization of the center of each spectrally separated PSF was determined by a Gaussian curve fitting using FISH_QUANT software (20) and then all centroids were allocated in pairs and distances measured by using MATLAB custom algorithms (see Materials and Methods). A vector transformation map (affine transformation matrix) was used to then correct the images for chromatic aberration. Arrows illustrate displacement vectors. Yellow spots illustrate corrected images. (C) Objective contour distortion map of chromatic aberration. The actual distortion determined by the vector map in (B) for the specific objective used in this study. The entire FOV is represented (in nm). Vectors indicated in black indicate chromatic shift direction and magnitude (Cy5 to Cy3). Bluer colors require minimal correction; warmer colors indicate major correction (in nm). (D) Percentage of colocalization between spectrally separated centroids before (black line) and after (red line) correction was applied to the entire FOV. (E) Distribution of observed distances of centroid pairs in two-color images after correction. Data (grey bars), Gaussian fit (red line), mean of distribution=7.86 nm±0.21 nm. Error, SEM.

FIG. 2A-2H. Determining significance of association between MCP and endogenous MBS-containing β-actin mRNA. (A) Schematic representation of smFISH-IF on β-actin mRNP: 24 MBS are present in β-actin 3′-UTR. Two MBS separated by linker regions (grey) are illustrated for simplicity. Cy3-labeled RNA FISH probes (MBS probes red stars) hybridized to linker regions as described (18) are depicted. The MCP fused to GFP (grey circles and green barrels respectively) is bound to the MBS as a dimer and can be detected by IF using antibodies against GFP and Alexa Fluor 647 (AF647) conjugated secondary antibodies (illustrated with green stars). (B,C) Representative smFISH-IF images from dissociated hippocampal neurons from MBS mice expressing MCP-GFP by lentivirus infection were probed for β-actin mRNA (B: MBS FISH probes, Cy3, red) or for CaMKII mRNA (C: CaMKII FISH probes, Cy3, red) and IF for MCP-GFP (GFP antibody, AF647, green). (B) A non-expressing MCP-GFP neuron only showed FISH signal (red). MAP2 is shown in blue as a dendrite marker.) (C) Images showed discrete fluorescent particles detected by both smFISH and IF throughout the dendrite that rarely overlap since the MCP doesn't bind CaMKII mRNA but binds β-actin mRNA with MBS in its 3′-UTR. (Scale bar, 5 μm.) Images are representative of 4 independent experiments, with over 15-20 dendrites observed in each experiment. (D) Schematic representation of a neuron and the super-registration method that measures the significance of each mRNA-protein pair (red and green dots, respectively and magnified). The circle represents the nearest red dot (mRNA). The simulation measures the frequency that the number of green dots (protein) within this area would fall within distances less than “d” by chance. (Inset: shaded area represents probability of chance association<0.1: the frequency for the illustrated pair based on 10,000 simulations). Every pair with this probability within 250 nm (the diffraction limit) is a single point in F and G. Complete data in FIGS. 8E and 8F. (E) Curve of association between an mRNA and a binding protein was calculated as the cumulative ratio of association for intermolecular distances (in the range between 0-to-250 nm) that were less than to a given observed distance. The ratio of association was calculated between the number of molecular pairs that can be found in proximity at each given nanometer of distance (and probability of chance association<0.1) and the total number of molecular pairs within 250 nm (see F and G). MCP-MBS (black line), MCP-CaMKII (dotted grey line). Red arrow shows the distance wherein the mRNA-protein association for MCP-MBS and MCP-CaMKII are maximally separated (optimal distance, OD=69 nm) (see Materials and Methods: ‘Measurement of association’ section). (F,G) Scatter plots show the probability of chance association between molecules for MCP-GFP and β-actin mRNA (MBS) in (F, MCP-MBS), and for MCP-GFP and CaMKII mRNA (CaMKII) in (G, MCP-CaMKII). ‘Box A’ (pink): the associated molecules that have a probability of chance association<0.1 and a distance less than to the OD of 69 nm (red vertical line, see E). These are the molecules that are physically likely to be in contact. ‘Box B’ (light yellow): molecules with a probability of chance association<0.1 but at distances greater than the OD and within the diffraction limit of 250 nm. These are the molecules that would be detected as positives by standard colocalization. The total number of intermolecular pairs in ‘Box A’=614 for MCP-MBS and 21 for MCP-CaMKII. The total number of pairs in ‘Box B’=120 for MCP-MBS and 111 for MCP-CaMKII. See also FIGS. 8E and 8F. (H) Distribution of observed distances for MCP-MBS (grey bars, Gaussian fit in red line) and MCP-CaMKII (MCP is bound to MBS on β-actin mRNA, black bars) after correction. Mean of observed distance was 34.58 nm±0.65 nm for MCP-MBS. Mean observed distance was 541.96 nm±8.14 nm for MCP-CaMKII (chance association, see also FIG. 8D). Error, SEM.

FIG. 3. Association between β-actin mRNA (MBS) and MCP as a molecular model mRNP. Schematic representation of overlapping red (RNA) and green (protein) diffraction-limited spots in a wide-field image and the molecular scale with nanometer precision of MCP-GFP and β-actin (MBS) interaction. By measuring and fitting a Gaussian curve to the PSF, the position in x, y and z of its center can be determined accurately with high spatial resolution (compare outer dotted line to inner dotted line). One Cy3-labeled MBS (red), MCP-GFP (green), primary antibody (IgY, light blue) and Alexa Fluor 647-labeled secondary antibody (IgG, purple) are depicted. The mean observed distance between labeled antibody and labeled RNA FISH probes is 34.58 nm (see FIG. 2H). The distance for MCP-GFP to β-actin mRNA is estimated in 7 nm. The drawing of the molecules was generated in PyMol software with the help of published structure data (22, 44).

FIG. 4A-4G. Association between ZBP1 and endogenous mRNA targets at molecular resolution. (A) Schematic representation of β-actin mRNA showing MBS and the zipcode (blue) bound by ZBP1 (light blue oval) in the 3′-UTR. Two MBS separated by linker regions (grey) are illustrated for simplicity. Cy3-labeled RNA FISH probes (MBS probes, red stars) and antibodies are also depicted. (B) Schematic representation of spinophilin mRNA showing two putative zipcodes (blue) bound by ZBP1 (light blue oval) in the 3′-UTR. Cy3-labeled RNA FISH probes (red star) and antibodies are also depicted. (C,D) Representative smFISH-IF image in dissociated hippocampal neurons from MBS mice expressing GFP-ZBP1 detected by GFP antibody (green) combined with smFISH for β-actin mRNA (MBS FISH probes, red) (C) and spinophilin mRNA (red) (D). Distal dendrites were analyzed where both smFISH and IF detected discrete fluorescent spots. Yellow arrowheads show sites of molecular interaction as defined by ‘Box A’ in FIG. 2 (probability of chance association<0.1 and OD=69 nm); white arrowheads show non-associated molecules as defined by ‘Box B’ in FIG. 2 (distances between OD and 250 nm). MAP2 is shown in blue as a dendrite marker. (Scale bar, 5 μm.) Images are representative of 5 for (C) and 2 for (D) independent experiments, with over 20 dendrites observed in each experiment. (E) Ratios of association for ZBP1-MBS and ZBP1-SPINO in neurons in comparison with the standard model MCP-MBS and MCP-CaMKII (negative control). Dotted red line indicates background association as defined by MCP-CaMKII. Error bar, SD. Unpaired t-test, **p<0.05; ***p<0.0001. (F,G) Distribution of observed distances for GFP-ZBP1 and β-actin mRNA (ZBP1-MBS) in (F) and GFP-ZBP1 with spinophilin mRNA (ZBP1-SPINO) in (G) after correction. Grey bars and red line, associated molecules as defined by ‘Box A’ (OD<69 nm); black bars, non-associated molecules as defined by ‘Box B’ (distances between OD and 250 nm). Mean of observed distance was 45.44 nm±1.80 nm for ZBP1-MBS in (F) and 41.00 nm±1.53 nm for ZBP1-SPINO in (G). Error, SEM.

FIG. 5A-5F. Validation of β-actin 3′-UTR affinity purification of associated proteins. (A) Schematic representation of β-actin 3′-UTR pull-down strategy. In vitro transcribed PP7-tagged zipcode-containing β-actin 3′-UTR RNA was incubated with MEF cell lysates, affinity purified on amylose magnetic resin and incubated with TEV protease either for 3 hrs or overnight (O/N) to identify protein components that interact with β-actin mRNA and ZBP1 protein. β-actin 3′-UTR containing one PP7 binding site (grey) bound by PCP fused to MBP (grey circles) and the zipcode element (red) and the coding region (light blue) are depicted. (B) Silver stained SDS-PAGE gel of proteins specifically bound to β-actin 3′-UTR RNA isolated from MEF extracts using either a control (‘C’, lanes 3 and 5) or β-actin 3′-UTR (lanes 4 and 6) as a bait. A list of proteins identified by LC-MS/MS is summarized in FIG. 11B. Red asterisk, PCP; black asterisk, MBP-PCP; double black asterisk, TEV protease. Molecular weight (Mr), kDa. Beads (‘B’, lane 1)=proteins remained bound to beads after TEV elution; Input (lane 2)=3 ug total protein; lanes 3-6=60% of pull-down eluates. (C) Western Blot analyses of indicated proteins in input and pull-down eluates upon TEV protease digestion for 3 hrs or overnight (O/N) as indicated. Molecular weight (Mr), kDa. Beads (‘B’, lane 1)=proteins remained bound to beads after TEV elution; Input (lane 2)=30 ug total protein; lanes 3-6=40% of pull-down eluates. The results shown are representative of 3 independent experiments. (D) RNA immunoprecipitation (RIP). Enrichment of endogenous β-actin (upper gel) and gapdh (lower gel) mRNAs in Dhx9 (Dx9), hnRNPAB (AB) and YBOX1 (YB1) immunoprecipitations (lanes 3-5) compared with IgG control (lane 6). A PCR reaction carried out without reverse transcriptase (-RT) is shown in lane 2. (E) Summary of association of the indicated mRNA and proteins by smFISH-IF in dendrites. Dotted red line indicates background association defined by MCP-CaMKII. Error bar, SD. Unpaired t-test, ****p<0.0001; *p<0.05; ns=p>0.05. (F) Venn diagram showing mRNA and protein association validated by both imaging and biochemistry approaches in this work. Asterisk, mRNA-protein associated validated by biochemistry in (16).

FIG. 6. Flow chart illustrating the steps to determine whether mRNA and protein molecules physically interact within cells.

FIG. 7A-7D. Mechanical shift correction for dual-color localization microscopy. (A) Schematic representation of the super-registration procedure for dual-color wide-field microscopy used to correct for microscope instability. In addition to the chromatic aberration correction, images were also corrected for mechanical shifts using an average displacement measurement calculated before and after image acquisition. Sub-diffraction fluorescent beads were imaged through z-stacks in Cy5 (green) and Cy3 (red) channels in between the registration of beads that were imaged in the same wavelengths (before and after registration). Localization of the center of each spectrally separated PSF was determined by a Gaussian fit using FISH_QUANT software (20) and all centroids were segregated by pairs and their distances measured using MATLAB custom algorithms. (B) Percentage of colocalization between centroids before (black line) and after (red line) correction was applied to the entire FOV. (C) Distribution of observed distances of centroid pairs in two-color images after correction. Data (grey bars), Gaussian fit (red line), mean of distribution=20.45 nm±0.22 nm. Error, SEM. (D) Scatter plot shows equidistant positions between localized centroids in Cy5 and Cy3 channels.

FIG. 8A-8H. MCP is associated with endogenous β-actin mRNA in MBS cells. (A,B,C) Representative smFISH-IF images in WT neurons (control): dissociated hippocampal neurons derived from WT mice expressing (A,B) or not expressing (C) MCP-GFP were probed for IF for MCP-GFP (GFP antibody, green) and smFISH using the following FISH probes: (A) MBS probes (Cy3, red), (B,C) β-actin ORF probes (Cy3, red). In WT neurons, β-actin mRNA did not have MBS in its 3′-UTR, thus, MCP-GFP did not bind the mRNA and it is retained in the nucleus due to a NLS signal. (A) No discrete fluorescent signal was detected in either channel. (B,C) Only fluorescent spots in smFISH channel were detected using β-actin ORF probes. MAP2 is shown in blue as a dendrite marker. (Scale bar, 10 μm.) Images are representative of 2 independent experiments, with over 20 dendrites observed in each experiment. (D) Distribution of observed distances for MCP-MBS (<50 nm, grey bars) and MCP-CaMKII (>150 nm, black bars). The higher observed distances between MCP and CaMKII mRNA suggest a random association. (E,F) Scatter plots showed the probability of chance association between molecules for MCP-GFP and β-actin mRNA (MBS) in (E, MCP-MBS), and for MCP-GFP and CaMKII mRNA (CaMKII) in (F, MCP-CaMKII). Boxes A and B are expanded in FIGS. 2F and g respectively for better visualization. (G,H) Histograms of signal intensity for MCP (G) and MBS (H). Grey bars, total population; red bars, physically associated mRNA and protein molecules defined by ‘Box A’.

FIG. 9A-9E. Super-registration as a molecular ruler. (A) Schematic representation of MBS-containing β-actin mRNA. Labeled RNA FISH MBS and ORF probes (red stars), MCP-GFP (grey circles and green barrels), and antibodies are depicted (green). Two MBS separated by linker regions (grey) are illustrated for simplicity. Distance between the stop codon to MBS is approximately 500 nucleotides (3′-UTR, shown in orange). (B) Ratio of association for MCP-GFP and β-actin mRNA in neurons comparing MBS or ORF probe sets (MCP-MBS and MCP-ORF, respectively) shows that OD with the ORF probes is 85 nm. Optimal distance (OD), nm. Error bar, SD. Unpaired t-test, ***p<0.001; ns=p>0.05. (C) Distribution of observed distances for MCP-MBS (light grey bars and black line) and MCP-ORF (black bars, and red line) shows the shift consistent with the increased distance from the MCP to the ORF. (D) Curve of association for MCP-MBS (black line), MCP-ORF (red line) and MCP-CaMKII (dotted grey line) demonstrates that the curves converge at 85 nm. (E) Normalization of the curves of association for MCP-GFP and β-actin mRNA (using MBS FISH probes, black line, MCP-MBS) and MCP-GFP and (β-actin mRNA (using ORF FISH probes, red line, MCP-ORF) (see D) reveals wherein the mRNA-protein association is maximally separated defining the optimal distance (OD). OD is 69 nm using MBS FISH probes (dotted black line, MCP-MBS) and 85 nm using ORF FISH probes (dotted red line MCP-ORF).

FIG. 10A-10F. Single-molecule FISH-IF shows association between ZBP1 and endogenous mRNA targets within neurons. (A) Field of view of the representative smFISH-IF image shown in FIG. 4C: dissociated hippocampal neurons from MBS mice expressing GFP-ZBP1 detected by GFP antibody (green) combined with smFISH for β-actin mRNA (MBS probes, red). ZBP1 is highly expressed in soma and proximal dendrites and less expressed in distal dendrites showing a puncta-like pattern. Only distal dendrites were analyzed where both smFISH and IF detected discrete fluorescent spots. smFISH-IF spot signals were dilated by 1 pixel for visualization. MAP2 is shown in blue as a dendrite marker. (Scale bar, 20 μm.) Inset is shown in FIG. 4C. (B,C) Scatter plot showed the probability of chance association for GFP-ZBP1 and β-actin mRNA (ZBP1-MBS) in (B) and GFP-ZBP1 with spinophilin mRNA (ZBP1-SPINO) in (C). Boxes A and B are expanded in (D) and (E) respectively for better visualization. ‘Box A’ (pink): the associated molecules that have a probability of chance association<0.1 and a distance less than to the OD of 69 nm (red vertical line). These are the molecules that are physically likely to be in contact. ‘Box B’ (light yellow): molecules with a probability of chance association<0.1 but at distances greater than the OD and within the diffraction limit of 250 nm. (F) Zipcode sequence alignment for β-actin and spinophilin 3′-UTRs as was described in (16). Spinophilin 3′-UTR showed two putative ZBP1 KH34 binding elements (zipcodes) (depicted in light blue) that have the same spatial arrangement as the unique bipartite zipcode in β-actin 3′-UTR (shown in red).

FIG. 11A-11J. Protein(s) associated with β-actin 3′-UTR by affinity purification. (A) Gene Ontology (GO) analysis and (B) Subcellular location and type of RNA-binding domain present in the new identified proteins associated with β-actin 3′-UTR by affinity purification coupled to LC-MS/MS analysis showed in FIG. 5. RRM, RNA recognition motif; KH, K Homology domain; CSD, cold-shock domain; DZF, domain associated with zinc fingers; RGG box, glycine-arginine-rich domain. N, nucleus; C, cytoplasm. (C) Western Blot analysis of indicated proteins in input and pull-down eluates. C=control RNA; β-act=β-actin 3′-UTR RNA. Molecular weight (Mr), kDa. (D-J) Observed distances for the indicated proteins and mRNAs shown in this study: (D) YBOX1-MBS; (E) Sam68-MBS; (F) hnRNPE2-MBS; (G) Dhx9-MBS; (H) hnRNPU-MBS; (I) hnRNPAB-MBS; (J) MCP-CaMKII. Grey bars and red line, associated molecules that have a probability of chance association<0.1 and a distance<OD (=69 nm) as defined by ‘Box A’; black bars, molecules with a probability of chance association<0.1 but at distances greater than the OD and within the diffraction limit of 250 nm as defined by ‘Box B’. See also histograms for MCP-MBS in FIG. 2H; ZBP1-MBS in FIG. 4F; and ZBP1-SPINO in FIG. 4G. Ratio of association was calculated as the ratio of the physically associated molecules as defined by ‘Box A’ to the total population of Boxes A and B.

FIG. 12A-12D. Protein(s) associated with β-actin 3′-UTR RNA bind to the zipcode region. (A) Schematic representation of the β-actin 3′-UTR and β-actin 3′-UTR containing a deletion of the zipcode sequence region (Δzip) RNAs used for pull-down. In vitro transcribed PP7-tagged zipcode-containing β-actin 3′-UTR and Δzip RNAs were incubated with MEF cell lysates and affinity purified on amylose magnetic resin in order to identify protein components that interact with β-actin mRNA and ZBP1 protein. (B) Sequence alignment for β-actin 3′-UTR and Δzip RNAs. (C) Silver stained SDS-PAGE gel of proteins isolated from MEF cell extracts using control (C), β-actin 3′-UTR (β-act) or Δzip 3′-UTR RNAs as a bait. WT=MEF cell extracts derived from wild type mice; KO=MEF cell extracts derived from ZBP1 KO mice. Molecular weight (Mr), kDa. (D) Western Blot analysis of indicated proteins in input and pull-down eluates. C=control RNA; β-act=β-actin 3′-UTR RNA. WT=MEF cell extracts derived from wild type mice; KO=MEF cell extracts derived from ZBP1 KO mice Molecular weight (Mr), kDa.

FIG. 13A-13F. Proteins associated with β-actin 3′-UTR by smFISH-IF. Representative smFISH-IF images in dissociated hippocampal neurons from MBS mice detected by smFISH for β-actin mRNA (MBS FISH probes, red) combined with IF for the indicated proteins (green): (A) YBOX1; (B) Sam68; (C) Dhx9; (D) hnRNPU; (E) hnRNPAB; (F) hnRNPU. Yellow arrowheads show sites of molecular interaction as defined by ‘Box A’ in FIG. 2 (probability of chance association<0.1 and OD=69 nm); white arrowheads show non-associated molecules as defined by ‘Box B’ in FIG. 2 (distances between OD and 250 nm). MAP2 is shown in blue as a dendrite marker. (Scale bar, 5 μm.) Images are representative of 2 for (A), 3 for (B), 3 for (C), 2 for (D), 2 for (E) and 3 for (F) independent experiments, with over 15-20 dendrites observed in each experiment.

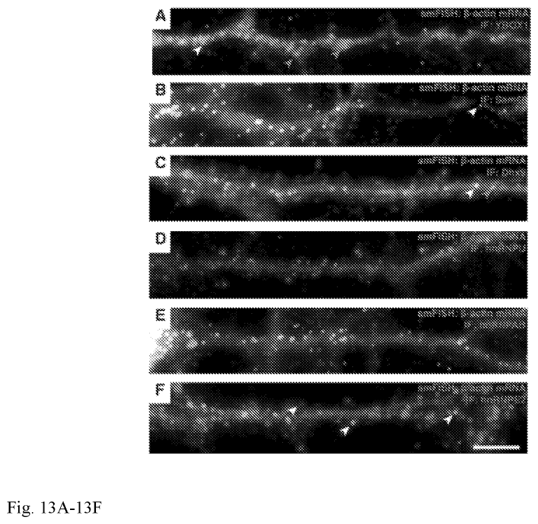

### DETAILED DESCRIPTION OF THE INVENTION

A method is provided for improving the performance of a fluorescence microscopy imaging system comprising an optical objective lens, a field of view, an imaging detector, and at least a first and a second fluorescent molecule, each of which fluoresces at a different wavelength than the other and each of which has a different excitation radiation peak than the other fluorescent molecule, the method comprising:
providing in a field of view of the fluorescence microscopy system a plurality of fluorescent beads capable of fluorescing at each of the different wavelengths of the first and second fluorescent molecules, wherein the beads have a diameter lower than a diffraction limit of the optical fluorescence microscopy system;
irradiating the plurality of fluorescent beads at an excitation radiation peak of the first fluorescent molecule and sequentially imaging the fluorescence of each of the plurality of beads within field of view of the fluorescence microscopy system and at a plurality of different z-dimension positions;
irradiating the plurality of fluorescent beads at an excitation radiation peak of the second fluorescent molecule and sequentially imaging the fluorescence of each of the plurality of beads within field of view of the fluorescence microscopy system and at a plurality of different z-dimension positions;
locating, from a point spread function of the fluorescence of each bead imaged at the excitation radiation peak of the first fluorescent molecule, the x,y coordinates of a centroid for each bead at each z-dimension position;
locating, from a point spread function of the fluorescence of each bead imaged at the excitation radiation peak of the second fluorescent molecule, the x,y coordinates of a centroid for each bead at each z-dimension position;
calculating, from a difference in the centroid x,y coordinates for each bead at the first and second excitation radiation peaks, a displacement vector for each x,y coordinate in the field of view at each z-dimension position, so as to thereby determine a displacement vector map for the optical objective of the fluorescence microscopy system;
applying the displacement vector map to imaging data obtained for the first and second fluorescent molecule so as to generate a fluorescence data image corrected for chromatic aberration in the optical objective of the fluorescence microscopy system.

In an embodiment, the beads are broad spectrum fluorescent beads. In an embodiment the broad spectrum beads are stained with four different fluorescent dyes of different excitation/emission peaks. In an embodiment the broad spectrum beads are stained with four different fluorescent dyes of the following excitation/emission peaks—360/430 nm (blue), 505/515 nm (green), 560/580 nm (orange) and 660/680 nm (dark red). In an embodiment, the beads are less than 250 nm in diameter. In an embodiment, the beads are 90-110 nm in diameter. In an embodiment, the beads are 100 nm in diameter

In an embodiment, the optical objective's chromatic aberration between the excitation radiation peak of the first and second fluorescent molecule is corrected for by applying an affine transformation. In an embodiment, the displacement vector map applied to imaging data obtained for the first and second fluorescent molecule so as to generate a fluorescence data image corrected for chromatic aberration is applied as an affine transformation matrix.

Also provided is a method of correcting for chromatic aberration in a fluorescence microscopy system comprising an optical objective lens, a field of view, an imaging detector, and at least a first and a second fluorescent molecule, each of which fluoresces at a different wavelength than the other and each of which has a different excitation radiation peak than the other fluorescent molecule, the method comprising:
providing in a field of view of the fluorescence microscopy system a plurality of fluorescent beads capable of fluorescing at each of the different wavelengths of the first and second fluorescent molecules, wherein the beads have a diameter lower than a diffraction limit of the optical fluorescence microscopy system;
irradiating the plurality of fluorescent beads at an excitation radiation peak of the first fluorescent molecule and sequentially imaging the fluorescence of each of the plurality of beads within field of view of the fluorescence microscopy system and at a plurality of different z-dimension positions;
irradiating the plurality of fluorescent beads at an excitation radiation peak of the second fluorescent molecule and sequentially imaging the fluorescence of each of the plurality of beads within field of view of the fluorescence microscopy system and at a plurality of different z-dimension positions;
locating, from a point spread function of the fluorescence of each bead imaged at the excitation radiation peak of the first fluorescent molecule, the x,y coordinates of a centroid for each bead at each z-dimension position;
locating, from a point spread function of the fluorescence of each bead imaged at the excitation radiation peak of the second fluorescent molecule, the x,y coordinates of a centroid for each bead at each z-dimension position;
calculating, from a difference in the centroid x,y coordinates for each bead at the first and second excitation radiation peaks, a displacement vector for each x,y coordinate in the field of view at each z-dimension position, so as to thereby determine a displacement vector map for the optical objective of the fluorescence microscopy system;
applying the displacement vector map to imaging data obtained for the first and second fluorescent molecule so as to generate a fluorescence data image corrected for chromatic aberration.

A kit is provided comprising a plurality of broad spectrum fluorescent beads and a non-transitory computer readable medium having instructions thereon for performing the methods described herein in a fluorescence microscopy imaging system.

Also provided is a method of detecting at least two co-localized fluorescent markers, wherein each of the two markers has a different emission spectrum, in a field of view of a fluorescence microscopy imaging system, the method comprising
subjecting an in vitro or in vivo system which has been preloaded with the two markers, wherein at least a portion of the in vitro or in vivo system is within the field of view of the fluorescence microscopy imaging system to irradiation at an excitation spectrum peak of each of the two different markers;
obtaining a fluorescence image for each two markers, when subjected to irradiation, with an optical objective of the fluorescence microscopy imaging system;
correcting the fluorescence images obtained for chromatic aberration of the optical objective at each of the different emission spectrums of the two fluorescent markers by a method described herein;
determining if the chromatic aberration-corrected fluorescence images show two colocalized different fluorescent markers, so as to thereby detect at least two co-localized fluorescent markers.

In an embodiment, each fluorescent marker is bound to a separate biological molecule. In an embodiment, the intermolecular distance for each of the two bound molecules is calculated from adjacent chromatic aberration-corrected fluorescent dye positions.

Also provided is a non-transitory computer-readable medium coupled to the one or more data processing apparatus coupled to a optical microscope fluorescence imaging system, the medium having instructions stored thereon which, when executed by the one or more data processing apparatus, cause the one or more data processing apparatus to perform a method as described hereinabove.

Also provided is a system for improving the performance of a fluorescence microscopy imaging system, comprising:
one or more data processing apparatus;
a graphical user interface; and
a non-transitory computer-readable medium coupled to the one or more data processing apparatus having instructions stored thereon which, when executed by the one or more data processing apparatus, and coupled to an optical microscope fluorescence imaging system, cause the one or more data processing apparatus to perform a method as described hereinabove.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, i.e., one or more modules of computer program instructions encoded on a non-transitory computer readable medium for execution by, or to control the operation of, data processing apparatus. The non-transitory computer readable medium can be a machine readable storage device, a machine readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database including a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Non-transitory computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

A non-transitory computer readable medium comprising instructions stored thereon for performing the methods described herein is also provided.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

Herein is provided a method to define colocalization precisely, as a non-random physical association of two labels at a resolution consistent with their molecular dimensions. Using fluorescent beads with a size below the diffraction limit of light to determine the characteristics of the optical objective and deriving a correction algorithm to co-register their centers of each Point Spread Function (PSF) at different wavelengths across the field of view (FOV) with nanometer precision, a process otherwise referred to herein as “super-registration” was developed.

The method was employed in tests using proteins known to bind mRNA in hippocampal neurons. Specifically, β-actin and spinophilin mRNAs were used and two proteins that have been previously shown to bind to them: an endogenous protein (zipcode-binding protein 1, ZBP1) (14-17) and an engineered protein that binds the MS2-binding sites (MBS) inserted into the 3′-UTR of β-actin mRNA (MS2 Capsid Protein, MCP) (18, 19). As a negative control, an mRNA was used that binds neither of these two proteins. These controls were used to develop a method to assess the significance of binding. It was tested whether RBPs isolated biochemically with a standard RNA pull-down met the binding test developed using this quantitative microscopic approach. The results demonstrate that by using standard light microscopy, one can identify with high probability whether these putative binding proteins actually interact with the mRNA, and how much. The approach is applicable to any two-labeled molecular species. Significantly, any standard fluorescence microscope can achieve this super-registration methodology by simple calibration of the objective lens coupled with subsequent image analysis. This approach provides the quality control for the information obtained from biochemistry techniques.

### Results

Super-Registration.

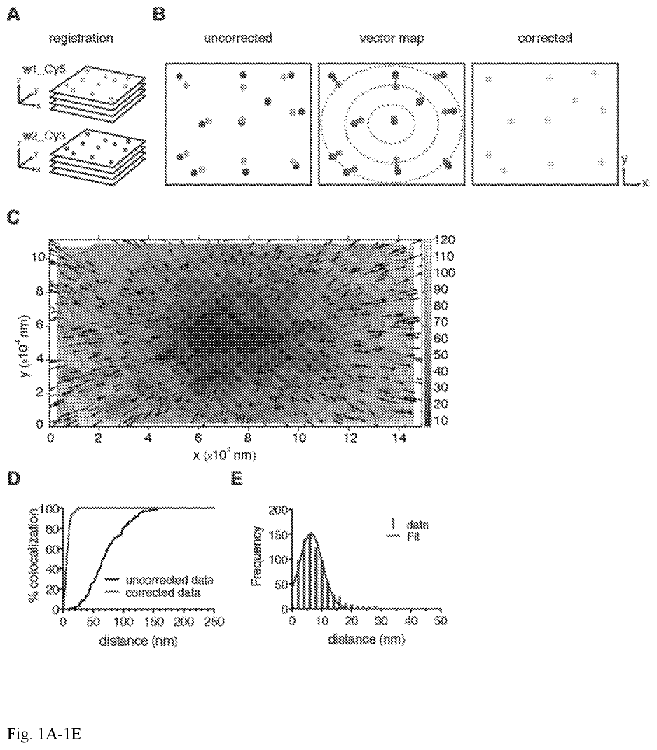

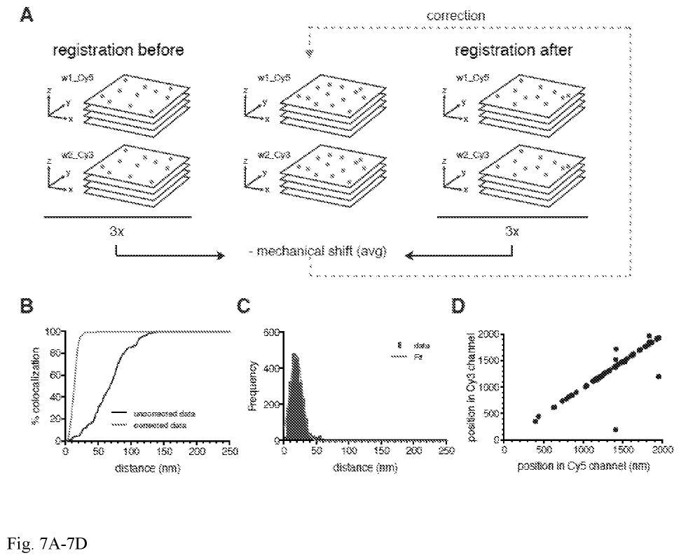

A new dual-color methodology was developed that reduced systematic errors limiting previous colocalization measurements by rigorously characterizing the microscope optics (see Materials and Methods). Sub-diffraction limited fluorescent beads were first imaged with a broad emission spectrum in z-stacks and then detected sequentially in Cy5 and Cy3 channels (FIG. 1A). The centroids of these beads were determined with sub-pixel precision (20). The displacement vectors were calculated between the centroid positions of each bead in the two channels as a function of its position in the field (FIGS. 1B and C). This process revealed that the chromatic aberration varied substantially from the center of the field to the edge (by as much as 120 nm, FIGS. 1C and D) due to the inability of the planapochromatic objective lens to correct across the entire field. In order to compensate for this, a transform was developed that reduced the error to less than 10 nm across the entire FOV (FIG. 1E and FIG. 7).

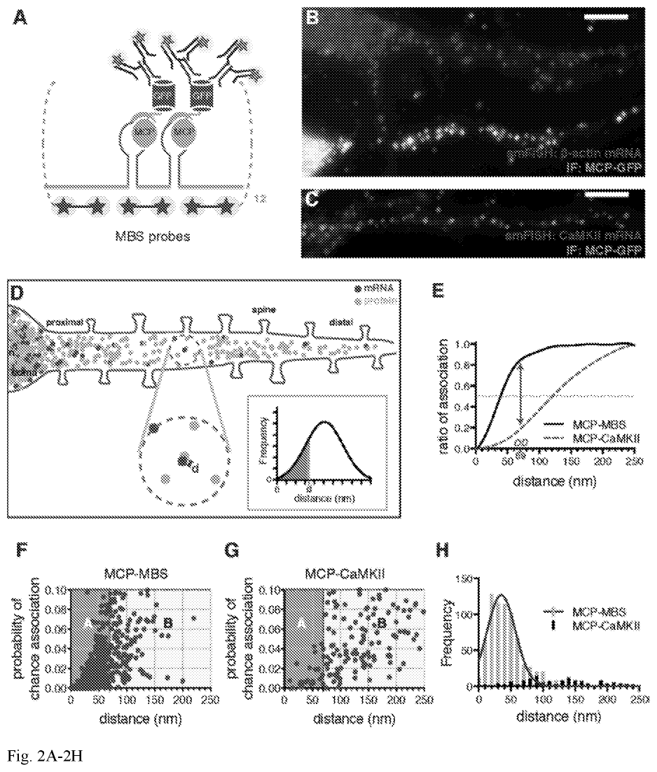

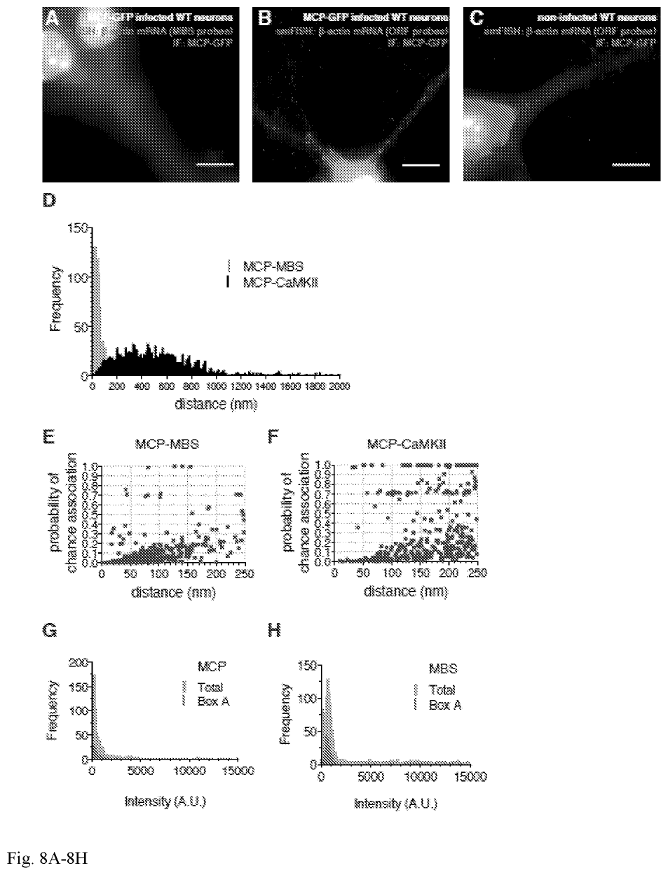

Imaging physical contact between MBS-containing β-actin mRNA and MCP. In order to provide a standard model system for calibration of protein binding, mRNA tagged with MBS (19) was used to visualize single mRNA molecules and their associated RBPs within fixed cells. Neurons derived from a mouse where 24 MBS were integrated into the 3′-UTR of the β-actin gene were cultured in vitro for 14-21 days (18). The fluorescent capsid protein MCP-GFP was introduced by lentivirus infection and specifically binds to MBS with high affinity (14, 21, 22). To confirm the intracellular association between MCP-GFP and single β-actin mRNA molecules within the cell, single-molecule fluorescence in situ hybridization (smFISH) was performed in combination with immunofluorescence (IF) in neurons (FIGS. 2A and B). It was found that MBS (β-actin mRNA) molecules overlapped with MCP signal, both of which appeared as diffraction-limited spots. Neurons derived from WT mice were used as a negative control for MCP association as they have no MBS. It was observed that the MCP-GFP in the nucleus (MCP has a nuclear localization signal) of these lentivirus-infected WT neurons, but no observation of any MCP-GFP spots in dendrites was made confirming that its association with the mRNA was MBS dependent (FIG. 8A-8C). These results indicate that both MCP-GFP (protein) and MBS (β-actin mRNA) are detected in close proximity within dendrites consistent with their expected intermolecular interaction.

Re-Defining Colocalization: Significance of RNA-Protein Association.

To ensure that the overlapping spots of smFISH to the MBS and IF to the MCP-GFP did not occur by chance, the likelihood of finding these two molecules in close proximity was measured. To address this, the negative control for RNA-protein association was included, in this case MCP-GFP and a dendritically localized transcript without MBS, CaMKII mRNA (FIG. 2C). After performing smFISH-IF for CaMKII and MCP-GFP, few events of close proximity between the two molecules was observed at distances less than 150 nm compared to MBS and MCP-GFP (FIGS. 2B and C and FIG. 8D). At increasingly larger distances (>150 nm) the spots are more likely to overlap by chance. In addition, any colocalization above 150 nm is not only a random event but occurs at a distance that is not relevant for physical contact.

The higher the local molecular density, the more likely that any colocalization could occur by chance and hence influence the level of specificity and significance for observed ‘colocalization’ events. Therefore, an analysis was designed that accounted for the local density around each of the associated pair of labeled molecules, in this case mRNA (red) and protein (green, FIG. 2D, expansion, Materials and Methods). The observed intermolecular distances for each pair were compared with a simulated Monte-Carlo random distribution of the two colors at similar concentrations. This provided a measurement to evaluate the significance compared to a randomized distribution. This probability of chance association was expressed when the simulation yielded a distance that was less than the observed distance (FIG. 2D, inset). The lower the probability of chance association, the higher the probability that the observed ‘colocalization’ reveals an intermolecular association that is statistically significant. Consistent with this, it was found that most MCP-GFP and MBS signals showed a high significance (probability of chance association<0.1). In contrast, most MCP-GFP and CaMKII signals did not show significant association (FIGS. 2F and G and FIGS. 8E and 8F).

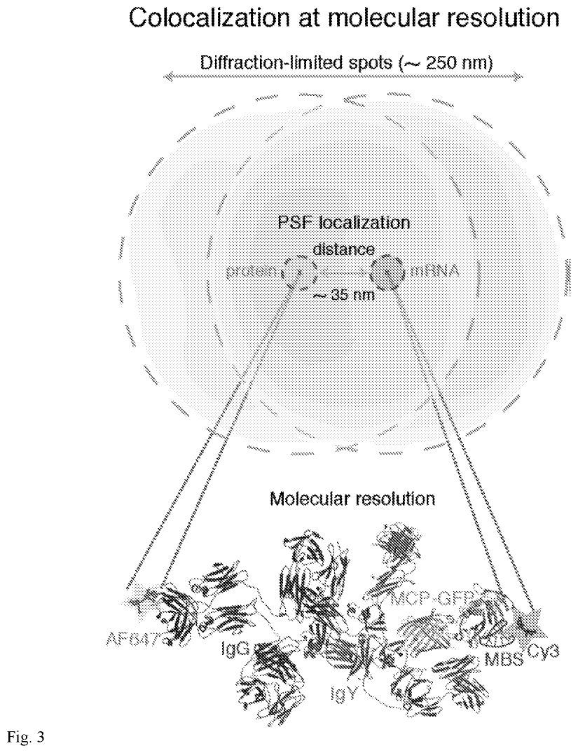

In order to obtain this probability measurement, association between the two molecules was calculated as a function of their distances apart for positive and negative controls (FIG. 2E and see Materials and Methods). For the positive control, eighty-five percent of the observed distances between the labeled probes to the MBS and the antibodies to the MCP-GFP were within 69 nm. In contrast, 15% of the observed associations in the negative control (MCP-GFP and the CaMKII probes) occurred at this distance (FIG. 2E, black line and dotted grey lines respectively). The 69 nm cut-off was determined to be the optimal distance (OD) between molecules where the difference between the detection of association for the positive control and detection of association for the negative control was the greatest (FIG. 2E, red arrows). Within this distance a probability of chance association less than 10% (<0.1) was defined that represented mRNA-protein molecules that were likely to interact (Box A′, FIGS. 2F and G, FIG. 8E-8H and Materials and Methods). In this analysis, it was found that there were mRNA-protein molecules with a probability of chance association less than 10% (because they were increased relative to the negative control) but were not in relevant proximity for a molecular interaction (i.e., distances ranging from OD=69 nm to 250 nm, ‘Box B’). For MCP-GFP and MBS, the mean observed distance was 34.58 nm±0.66 nm (FIG. 2H). This measurement includes the distance from the labeled antibodies detecting MCP-GFP to the labeled oligonucleotide probes used to detect β-actin mRNA (using MBS FISH probes). A molecular model for the physical association of MCP-GFP and MBS using available crystal structures in PyMOL indicated that the antibodies positioned the fluorescent label approximately 25 nm away from the MCP-GFP. This model supports the conclusion that standard wide-field microscopy is capable of resolving a bona fide mRNA-protein complex (FIG. 3).

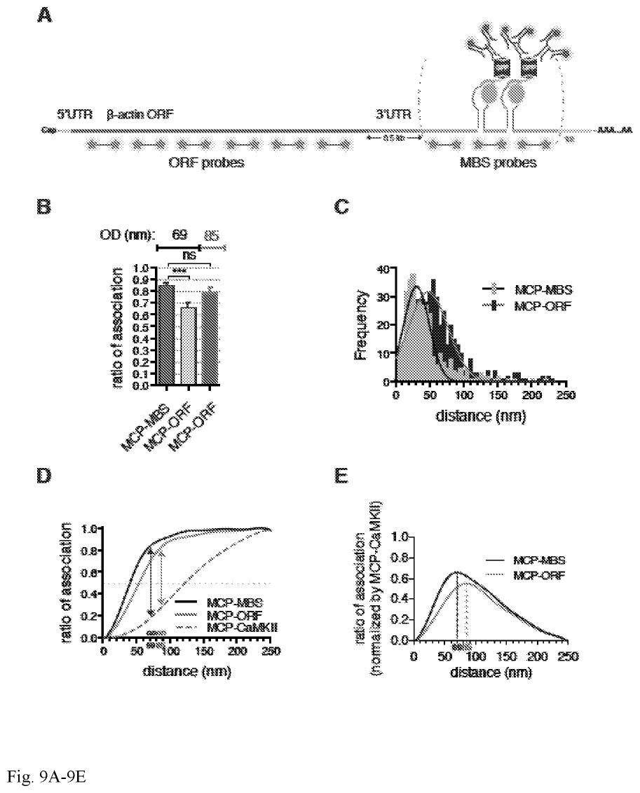

The precision of the registration demonstrated that physical distances between the location where the protein is positioned relative to the FISH probes could be mapped within 10-to-20 nm, depending on their separation, demonstrating that this approach can serve as a “molecular ruler” (see FIG. 9).

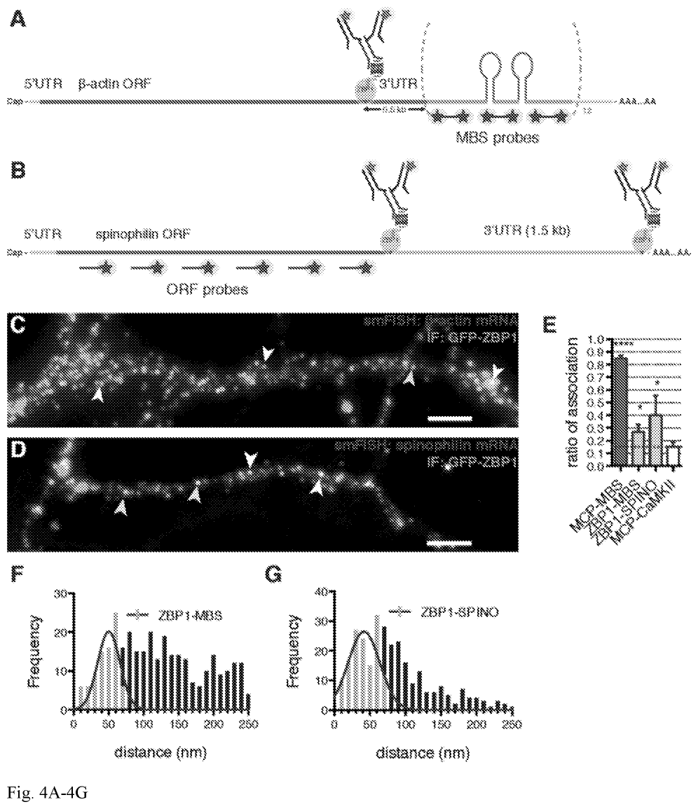

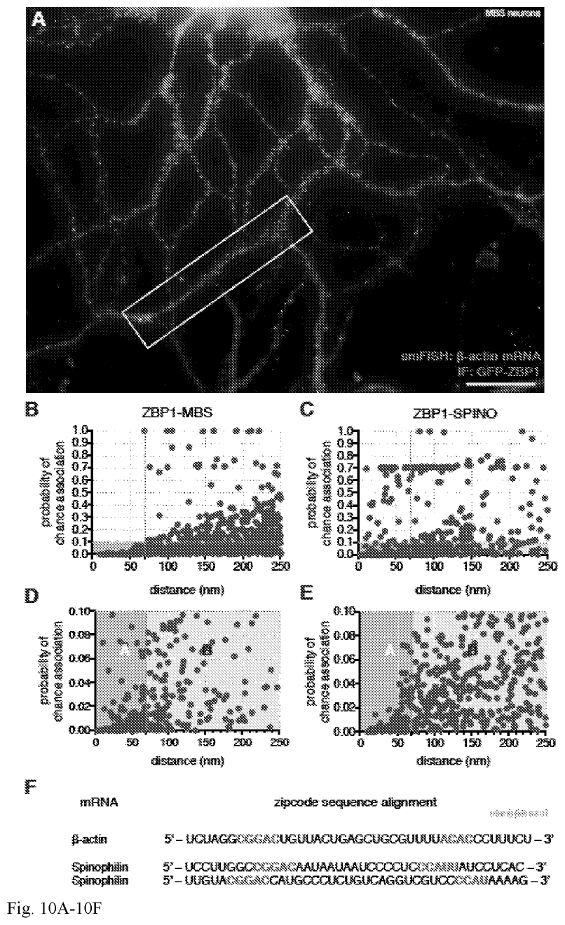

Application to the interaction between ZBP1 and its mRNA targets. This analytical technique was then tested on a bona fide endogenous complex: the well-characterized interaction between β-actin mRNA and ZBP1, the protein that binds to its bipartite zipcode sequence element present in the 3′-UTR (14, 16). MBS neuronal cultures infected with lentivirus encoding GFP fused to ZBP1 showed discrete particles along mature dendrites, reminiscent of dendritically transported mRNA granules with different sizes and signal intensities (FIGS. 4A and C and FIGS. 10A and 10F). Analysis of the images revealed that the overlap between β-actin mRNA (FISH signal) and ZBP1-GFP (IF signal) was 27% (FIGS. 4E and F and FIGS. 10B and 10D). This association of ZBP1-GFP with the mRNA is less than MCP-GFP, which has essentially a longer off rate. Besides β-actin mRNA, other targets for ZBP1 have been described (16). For instance, spinophilin, a zipcode-containing mRNA, was enriched in pull-down experiments for ZBP1 from brain extracts and localized to mature dendrites dependent on ZBP1 (16). In support of this, ZBP1-GFP was observed in close proximity with spinophilin mRNA within mature dendrites (FIGS. 4B, 4D, 4E and 4G and FIGS. 10C, 10E and 10F). The findings showed one population of interacting molecules (0-to-69 nm) and other from 69-to-100 nm consistent with this mRNA having two putative zipcodes (FIGS. 4B and 4G and FIG. 10F). The ZBP1-GFP molecules bound to spinophilin mRNA molecules at OD<69 nm was greater than those bound to β-actin mRNA (using MBS FISH probes) (FIG. 4E). These results demonstrate that this imaging method has the resolution to determine where in the dendrite a direct interaction occurs between an RBP such as ZBP1 with its mRNA targets and its relative degree of association compared to MBS-MCP.

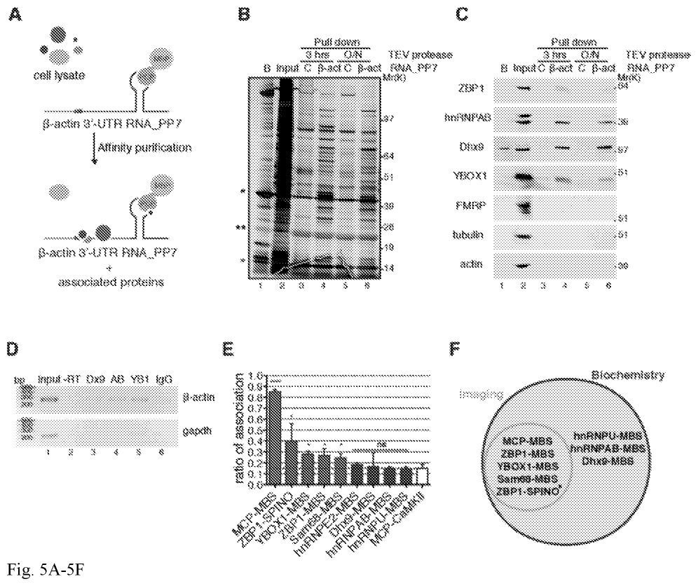

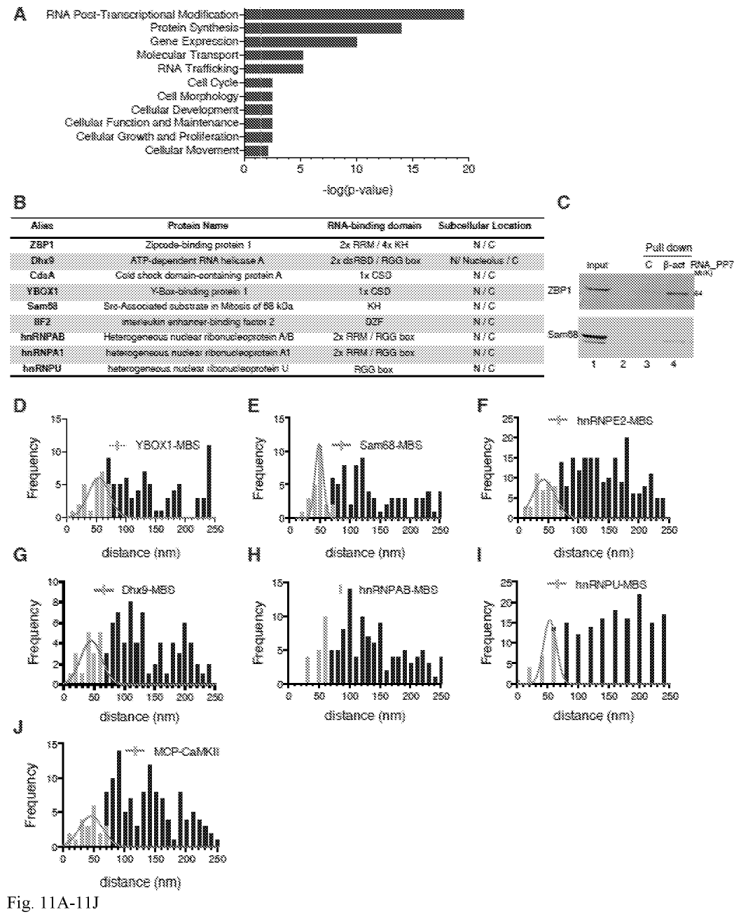

Validation of novel β-actin mRNA associated factors. To evaluate the efficacy of this approach to validate putative RNA-protein interactions, we isolated additional binding proteins for β-actin mRNA by a typical pull-down assay. By using in vitro transcribed PP7-tagged zipcode-containing β-actin 3′-UTR RNA as bait, stably associated proteins were captured from mammalian cell extracts (FIGS. 5A and B). Proteins specifically bound to β-actin 3′-UTR RNA were eluted, separated by SDS-PAGE and analyzed by Liquid Chromatography-Mass Spectrometry (LC-MS/MS). Gene ontology (GO) analysis revealed that proteins found associated with β-actin 3′-UTR were principally involved in RNA post-transcriptional modification, protein synthesis, gene expression and RNA trafficking functions (FIGS. 11A and 11B). In addition to ZBP1, hnRNPs AB, A0, A3, A1, L, D, DL, UL1, U, Q1 (Syncrip), R, Y-Box binding protein 1 (YBOX1), Cold shock domain-containing protein A (CsdA), ATP-dependent RNA helicase A (Dhx9), IMP2, IIF2, Staufen 1 & 2, PABP1, Src-associated in mitosis 68 kDa (Sam68), Myelin expression factor 2-like (Myef2), UPF1, eIF3 and several SR proteins we found, as well as the motor related protein MRLC2 (Myosin Regulatory Light Chain 2).

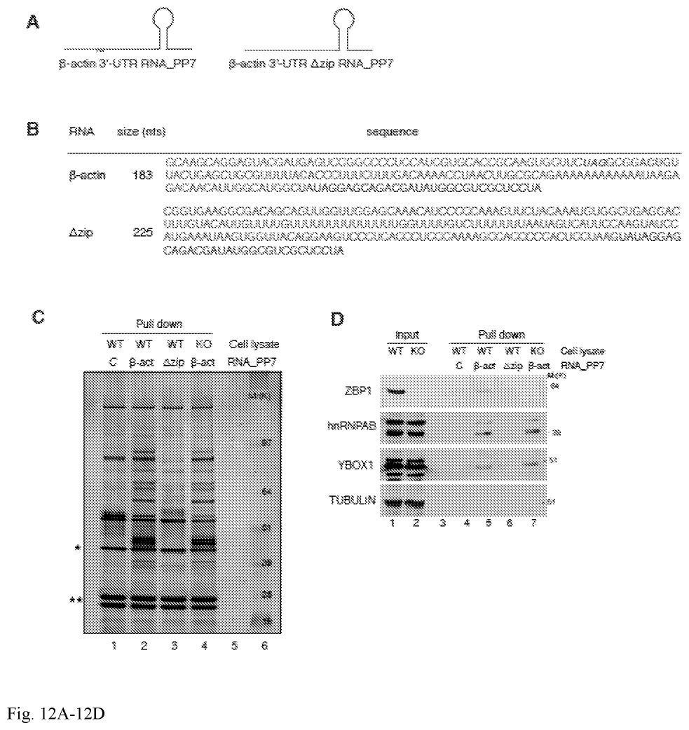

The association between β-actin 3′-UTR RNA and novel proteins identified was confirmed by standard biochemical techniques such as Western blot (FIG. 5C and FIG. 11C) and RIP (RNA immunoprecipitation) (FIG. 5D). ZBP1, hnRNPAB (23), Dhx9, YBOX1 and Sam68 (24) showed a significant interaction with β-actin 3′-UTR RNA in comparison with the control RNA. Non-RNA binding proteins such as tubulin or actin were not detected in pull-down eluates indicating enrichment in specific binders. FMRP, a prominent neuronal mRNA binding protein (25), was not detected either by LC-MS/MS or Western blot analysis. While Western Blots in FIG. 5C highlighted the specificity of protein-RNA interactions found by LC-MS/MS, endogenous β-actin mRNA was found in eluates of immunoprecipitations carried out by specific antibodies against Dhx9, hnRNPAB and YBOX1 (FIG. 5D). Binding of ZBP1, hnRNPAB, YBOX1 and Sam68 was precluded when a β-actin 3′-UTR RNA containing a deletion of the zipcode sequence region was used suggesting they bound to the zipcode, or were part of a zipcode binding complex (FIG. 12).

Finally, we tested the identified RNA-protein associations by super-registration microscopy. YBOX1, Sam68, hnRNPE2, hnRNPU, hnRNPAB and Dhx9 immunofluorescence combined with smFISH for β-actin mRNA (using MBS FISH probes) was performed in fixed neurons and intermolecular distances were calculated (FIGS. 5E and F, FIGS. 11 D-J and 13). RNA-protein associations ranged from 10% to 40% for all the identified factors analyzed with β-actin mRNA in hippocampal dendrites (FIG. 5E). ZBP1, YBOX1 and Sam68 were associated with β-actin mRNA, however Dhx9, hnRNPE2, hnRNPU and hnRNPAB were non-specific in their interactions, similar to the association of CaMKII (15%). Similar molecular conformations and dye orientations were assumed for each pair and the OD less than 69 nm previously determined was used. Therefore, two-color imaging can critically evaluate whether single molecules of mRNA make bona fide physical contacts with putative binding proteins.

### Discussion

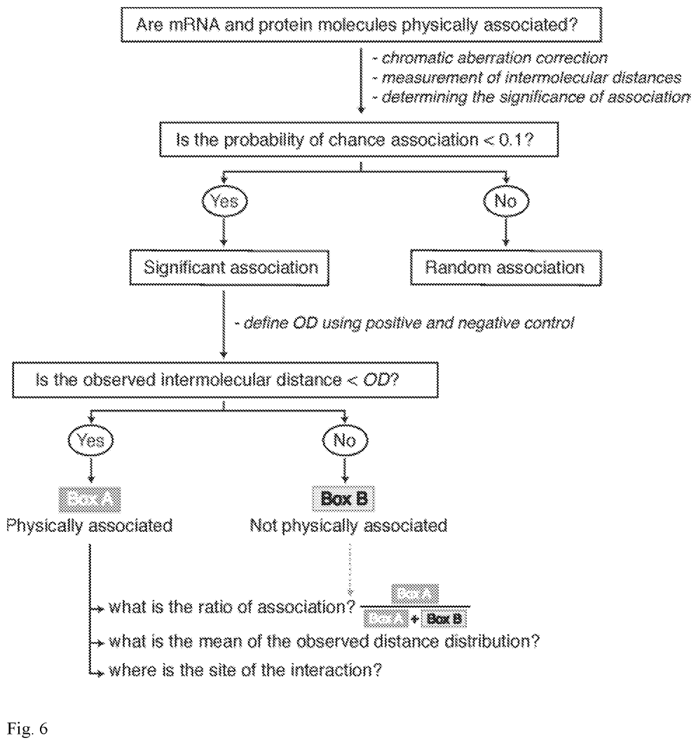

In this study an approach is provided to ascertain the physical interaction between single mRNAs and binding proteins in situ in single cells using standard wide-field microscopy. A flow chart of an exemplary method is illustrated in FIG. 6. This imaging method extends biochemical-based studies on RNA-protein interactions by providing spatial information about where in the cells these interactions are likely to occur. This is especially important in neurons, in which RNA regulatory mechanisms play an essential role in the regulation of localized gene expression.

The analysis of colocalization has as its basis the likelihood of finding two molecules in close proximity. For instance, colocalization is deduced by the merging of two colors (e.g., a yellow spot when comparing red and green pseudo-colors). However this may not indicate real association between molecules. First, the resolution may not be sufficient to determine the true distance between the colors. Second, the overlap may have occurred by chance dependent on the concentrations of each of the molecules. By this same reasoning, two molecules may be colocalized even if a merged signal is not apparent, due to chromatic aberration or disparities in the brightness of each component. In this work, we have developed a quantitative image acquisition and analysis method that measures the distance between labeled molecules and the likelihood of their physical association independent of their intensities.

Various statistical methods have been proposed to address colocalization using single-molecule imaging. A dominant method is the Ripley's K function method (reviewed in (26)), which tests spatial randomness through the computation of its quantiles. This method and its derivatives have been developed to create a fast and robust statistical test. However, this approach is limited since the region of interest requires straight lines at its edges to account for edge-effect biases, and may not be as accurate as the more computationally expensive Monte-Carlo simulation. Since neuronal structure is highly irregular and small sets of pairing events require quantitative characterization, we centered our study on the interaction between individual mRNAs and proteins without analyzing the global spatial molecule distribution through a region of interest (ROI). Therefore, the imaging analysis described here allows an objective quantification of the probability of molecular association and it is independent of the molecular density within the cell.

Chemical and UV crosslinking followed by RNA-sequencing after immunoprecipitation (e.g., CLIP) has been used to identify putative mRNA-protein associations (27-32). However, while these techniques show that these molecules can interact, it does not provide evidence of a stable in vivo complex; the molecules may come in contact transiently upon cell disruption or be artificially stabilized by crosslinking (33, 34). In contrast, imaging at the single-molecule and cellular level provides evidence of a biologically relevant interaction. In addition, the percent binding can be represented spatially in unmodified cells: where in the cell this binding is likely to occur.

This imaging method can characterize and validate novel protein components of a specific mRNP. In addition to the well-known ZBP1, other proteins were found that bound to the zipcode-containing β-actin 3′-UTR using a PP7 stem-loop to pull-down the RNA. From the list of protein candidates that bound the β-actin 3′-UTR, the presence of YBOX1, hnRNPAB and Dhx9 were consistent with its presence in ZBP1/IMP1 RNP granules (35, 36). Sam68 has also previously been found to bind to β-actin mRNA in neurons and regulate its translation (24, 37, 38). More importantly, the approach will be instrumental in ruling out false positive associations. For instance, hnRNPAB has been shown to bind AU-rich response elements commonly present in 3′-UTRs (39-42) and we find it associated with β-actin 3′-UTR by affinity purification. However this approach reveals that hnRNPAB and β-actin mRNA do not interact except by chance in dendrites. Similarly, hnRNPU, and Dhx9, an RNA helicase mostly enriched in the nucleus, also do not associate with β-actin mRNA except by accident in dendrites in contrast to results that suggested specific binding using biochemical techniques (FIG. 5). It should be noted, however, that the observations do not negate the possibility of a physiologically significant effect of these proteins, since a transient interaction may be sufficient for a protein to modify an RNA, or promote formation of a complex, even if the interaction occurs statistically by chance. Nonetheless, this method clearly identifies proteins (ZBP1, YBOX1 and Sam68) that are stably associated with β-actin mRNA at intermolecular distances below 69 nm, the threshold for distinguishing physically meaningful interactions. However, it is also possible that proteins in a large complex (>69 nm) may be associated but not in physical contact with the mRNA. In addition, the association of ZBP1-GFP with β-actin mRNA may be underestimated because there was competition with the endogenous ZBP1 for β-actin mRNA binding. ZBP1 also dissociates from the mRNA depending on its phosphorylation (15, 43). Finally, the detection of the ZBP1-GFP by antibodies would be less efficient than direct labeling of mCherry-ZBP1 in cells derived from a knockout mouse, where all ZBP1 is labeled (43).

Identifying bona fide RNA-protein associations in situ is important for investigating their roles in a variety of molecular and subcellular events, such as local translation in synaptic plasticity. The RNA-protein interactome can be explored with the methodology described here. Single-molecule FISH-IF can be generally applied to any combination of mRNA and binding protein(s) allowing single mRNP complex observation at cellular sites of mRNP assembly. Notably, endogenous mRNAs and proteins can be directly investigated by using RNA FISH probes and antibodies commercially available, without genetic manipulation of the cells. Importantly, this approach can be achieved by simple fluorescence microscopes and does not require laser illumination, EM-CCD cameras, long imaging acquisition times, deconvolution or image reconstruction. Thus, this imaging method will be an essential technique to complement biochemical studies since the spatial relationship within the cell is preserved.

### Materials and Methods

Mouse Hippocampal Neuron Culture.

Animal work was performed in accordance with IACUC protocols at Albert Einstein College of Medicine. Post-natal mouse hippocampal tissue was isolated from homozygous MBS knock-in (18) newborn pups (P0-P1). Hippocampi were placed in 0.25% trypsin for 15 minutes at 37° C. Tissue was triturated and plated onto poly-D-lysine (Sigma) coated glass-bottom dishes (MatTek) at 45,000 cells per dish and cultured in Neurobasal A media (Life Technologies) supplemented with B-27 (Life Technologies), GlutaMax (Life Technologies) and primocin (InvivoGen). Hippocampal neurons from wild type (WT) mouse embryos (E18) (BrainBits, LLC) were prepared as above. Dissociated mouse hippocampal neurons were infected with lentivirus expressing MCP-GFP or ZBP1-GFP at 5 days in vitro.

Single-molecule FISH in combination with immunofluorescence (smFISH-IF). Combining smFISH with IF required multiple conditions to accommodate both reagents. Fixation, permeabilization and staining: mouse postnatal hippocampal neuronal cells infected on DIVS with lentivirus encoding for tandem-dimer MCP-GFP were fixed at DIV 14-21 with ice-cold 4% (vol/vol) paraformaldehyde and 4% (wt/vol) sucrose in 1×PBS-MC (1×PBS supplemented with 1mM MgCl₂ and 0.1 mM CaCl₂) for 20 minutes; quenched in 50 mM Glycine, and permeabilized with ice-cold 0.1% Triton X-100 (Thermo Scientific, #28314) and 0.5% UltraPure BSA (Life Technologies, AM2616) in 1×PBS-MC for 15 minutes. After incubation with 10% formamide, 2×SSC, 0.5% UltraPure BSA in RNAse-free water for 30 minutes at room temperature, cells were incubated for 3 hours at 37° C. with either 10 ng (Invitrogen) or 50 nM (Stellaris RNA FISH probes, Biosearch Technologies) labeled mix probe sets and primary antibody against GFP from Ayes Labs, Inc. (GFP-1010) at 1/5000 dilution in Hybridization Buffer (10% formamide, 1 mg/ml E. coli tRNA, 10% dextrane sulfate, 20 mg/ml BSA, 2×SSC, 2 mM Vanadyl Ribonucleoside Complex (VRC), 10 U/ml Superase. In (Ambion) in RNAse-free water). Then, cells were quickly washed and incubated twice with Alexa Fluor 647 conjugated secondary antibody (Life Technologies) at 1/1000 dilution in 10% formamide, 2×SSC in RNAse-free water for 20 minutes at 37° C. After four 2×SSC washes DNA was counterstained with DAPI (0.5 μg/ml in 2×SSC; Sigma) and after a final wash, cells were mounted using ProLong gold antifade reagent (Life Technologies). smFISH-IF spot signals were dilated by 1 pixel for visualization.

Microscope Set Up.

Images were taken using a upright, wide-field Olympus BX-63 microscope equipped with a SuperApochromatic 60×/1.35 NA Olympus objective (UPLSAPO60XO), X-Cite 120 PC lamp (EXFO), ORCA-R2 Digital Interline CCD camera (C10600-10B, Hamamatsu) mounted using U-CMT and 1X-TVAD Olympus c-mount adapters and zero pixel shift filter sets: DAPI-5060C-Zero, FITC-5050A-Zero, Cy3-4040C-Zero and Cy5-4040C-Zero from Semrock. The resulting image pixel size was 107.5 nm and the z-step size (along the optical axis) used for all optical sectioning acquisition was 200 nm. To position the specimen more accurately along the optical axis (in z) and to minimize mechanical vibration, a PZMU-2000 Piezo-Z Top Plate from Applied Scientific Instrumentation was used. A webcam was used to monitor the automated acquisition remotely to avoid turbulence and temperature fluctuations in the microscope environment. To improve optical stability, we used a vibration isolation table (TMC) and ensured that airflow did not affect the microscope stand. The environmental control system maintained constant temperature (20° C.±1° C.) and low humidity (35%±5% relative humidity) during a given experimental day. Metamorph software (Molecular Devices) was used for controlling microscope automation and image acquisition.

Super-Registration.

The objective's chromatic aberration across the entire FOV was compensated for using a map that described the optical distortion as a function of position by observing sub-diffraction limit sized fluorescent beads that have broad emission spectra (TetraSpeck fluorescent microspheres, 100 nm diameter, Life technologies). Multiple fields of beads (n=760 beads) were imaged in three dimensions sequentially in Cy5 and Cy3 channels. Then, centroids of the PSF of the beads were localized with sub-pixel precision in each channel (see Single-molecule localization). The Cy5 channel centroid positions in x and y were compared to the Cy3 channel centroid positions in x and y, and the displacement vectors between the centroid positions of each bead in the two channels were calculated. The displacement vectors were determined in each orthogonal axis independently as a function of the position in the FOV. The objective's chromatic aberration between Cy5 and Cy3 was compensated using an affine transformation. A detailed description of the super registration can be found hereinbelow.

Bead Preparation.

Beads were diluted with distilled water and uniformly suspended by sonication before they were loaded to a poly-L-lysine coated coverslip. Once the beads settled and dried, Prolong Gold mounting media reagent (Life Technologies) was added, left overnight on a level surface in the dark and then the coverslip was sealed with nail polish.

Objective Testing.

The optical calibration on 6 matched objectives acquired from Olympus was tested. All of these 60× objective lenses showed unique variations in their chromatic aberrations. Each objective lens was unique in its performance characteristics having its own ‘fingerprint’ for optical distortion across the FOV. The objective that required the least total chromatic correction in our optical path was used for this study (UPLSAPO60XO, 4K020 serial number).

Single-Molecule Localization.

To determine the centroid position of single molecules FISH_QUANT software (20) was used (free, available online). Briefly, after background subtraction, the software fitted a 3D Gaussian function to the PSF of the single-molecule, which yielded centroid coordinates in each channel with sub-pixel accuracy (<20 nm). Auto-fluorescent and non-specific signal were excluded by thresholding the intensity and by the width of the 3D Gaussian curve.

Measurement of Intermolecular Distances and Determining the Significance of Association.

Software was written in MATLAB (MathWorks) to identify centroid pairs using nearest-neighbor algorithm (pairing), measure intermolecular distances (in nm) and provide significance of association for each pair of molecules between the two channels. The method determined the probability of chance association for each intermolecular pair based on the intermolecular distances observed and the local molecular density within the cell.

Measurement of Association.

The following procedure determined the largest distance that two molecules could be separated and still be considered physically associated. First, the intermolecular distances and significance of association from a positive and negative control were calculated, in this case MCP-GFP and MBS (MCP-MBS) and MCP-GFP and CaMKII (MCP-CaMKII), respectively (as described above in ‘Measurement of intermolecular distances and determining the significance of association’ section). Then, the molecular pairs that exhibited the most significant probability of chance association (<0.1) and that had a intermolecular distance<250 nm (diffraction limit) were selected. The cumulative ratio of association for intermolecular distances (in the range between 0-to-250 nm) that were less than or equal to a given observed distance was plotted (for both positive and negative controls separately) (FIG. 2E). The distance wherein the difference was the highest between the detection of association for MCP-MBS (‘signal’) and detection of association for MCP-CaMKII (noise') defined the optimal distance (OD), in this case 69 nm (FIG. 2E, red arrows). At the OD, the signal-to-noise ratio is maximized. Thus, we used the distance of 69 nm as the OD in the analysis of RNA-protein interaction, unless otherwise noted. Only the molecular pairs with probability of chance association<0.1 and intermolecular distances<OD were considered associated and defined the population of pairs included in ‘Box A’ (FIGS. 2F and G). ‘Box B’ was defined as the population of molecular pairs with probability of chance association<0.1 but at intermolecular distances in the range from the OD to 250 nm. Finally, the ratio of association between molecules of mRNA and protein was expressed as the ratio of the population of ‘Box A’ to the population of Boxes A and B combined. OD is dependent on both the positive and negative control analyzed.

The interacting labeled-molecules included in ‘Box A’ showed intensities that were representative of the total molecular population analyzed (FIGS. 8G and 8H). This indicates that this imaging is able to identify bona fide mRNA-protein associations based on the spatial position of their fluorophores, independent of their intensities.

Imaging Analysis Software.

All image analysis was performed with existing software packages and custom algorithm programs written in MATLAB (MathWorks). The code provides (i) chromatic aberration and mechanical shift corrections (super-registration); (ii) identification of centroid pairs (pairing) and measurement of intermolecular distances (in nm); (iii) evaluation of the probability of chance association; and (iv) ratio of association as described in this work. The software is able to read FISH_QUANT (20) detected spot files (version 3D_v1) and import all the centroid positions in x and y along with the corresponding ROI chosen. It can be imported as many ROIs as the image has at once. The code (version 1.0) is available online through our website, open-access for anyone to use without restriction.

PP7-Based RNA Affinity Purification (pull-down).

Amylose magnetic resin (NEB) was washed twice and incubated with recombinant purified protein MBP-PP7 and pre-heated PP7-β-actin 3′-UTR RNA (ratio 1:1) in binding buffer (20 mM Tris pH 7.2, 200 mM NaCl, 1 mM EDTA pH 8.0, 1 mM DTT, 0.01 mg/ml tRNA, 0.01% IGEPAL) for 1 hour at 4° C. with constant rotation. The pull-down was then performed by adding cell extract aliquots (5-30 mg total protein) supplemented with 100 mM NaCl and 0.01 mg/ml tRNA to the RNA immobilized to the beads through the MBP-PP7 protein followed by incubation at 4° C. for 2 hours with constant rotation. Total protein aliquots used in pull down procedures varied and are listed in Figure legends. 1.5-ml non-stick microcentrifuge tubes were used when working with small volumes or 15-mL sterile polypropylene centrifuge tubes with larger volumes. Following pull-down, the magnetic beads were washed 5 times (1-ml volume washes) with ice-cold wash buffer (20 mM Tris pH 7.2, 200 mM NaCl, 1 mM EDTA pH 8.0, 1 mM DTT, 0.01% IGEPAL) and transferred to a new tube in last wash step. For RNP complexes elution from the beads, TEV protease was added to the beads followed by 3 hours of incubation at 4° C. with rotation. Alternatively, 500 μl of 0.5 M NH₄OH supplemented with 0.5 mM EDTA pH 8.0 was added to the beads followed by 20 minutes incubation at room temperature with rotation. After beads were removed, eluate fractions were lyophilized in the speed vac for at least 4 hours at room temperature. For protein analysis using SDS-PAGE, the eluates were incubated with appropriate volume of 4× protein sample buffer (Invitrogen) supplemented with 50 mM DTT and heated at 70° C. for 10 minutes.

### Supplemental Methods

Super-registration. The premise of super-registration is that we need to compensate for the intrinsic inability of optics to correct completely for chromatic aberration and for other factors that influence the optical path in a way that interferes with the fidelity of detecting centroid positions using single-molecule localization techniques. It was found that minimizing the influence of and compensating for these aspects was essential for achieving exquisite alignment of multiple fluorescence channels to ten nanometer precision.

Chromatic aberration is a common optical problem that occurs when wavelengths of different color are focused at different positions in the focal plane. Using high quality, super planapochromatic objectives does minimize this optical distortion. However, these lenses still do not provide a perfectly corrected image from edge to edge of the field of view (FOV) of a typical detector (144.48 μm×110.08 μm). The objective's correction works best just at the center of the FOV. Therefore, to compensate for the objective's chromatic aberration across the entire FOV, we mapped the optical distortion as a function of position by observing sub-diffraction limit sized fluorescent beads that have a broad emission spectrum (TetraSpeck fluorescent microspheres, 100 nm diameter, Life technologies). Multiple fields of beads (n=760 beads) were imaged in three dimensions sequentially in the Cy5 and Cy3 channels FIG. 1A). Then, centroids of the Point Spread Functions (PSF) of the beads were localized with sub-pixel precision in each channel (see Materials and Methods: ‘Single-molecule localization’ section). The Cy5 channel centroid positions in x and y were compared to the Cy3 channel centroid positions in x and y, and the displacement vectors between the centroid positions of each bead in the two channels were calculated (FIG. 1B). For simplicity of analysis, only the x-y plane was taking into account since the imaging was done with z-sectioning. Ideally, the displacement vectors would be zero across the entire FOV. However, we observed an offset caused by chromatic aberration. We determined the displacement vectors in each orthogonal axis independently as a function of the position in the FOV. For the objectives we used, the function that best fitted the displacement data for the x and y-axes was a plane. This was consistent with the observation of a radial optical aberration in which the magnitude of the distortion increased as a function of the position from the center of the FOV (where centroid positions in x and y were practically identical in the two channels) towards the edges (FIGS. 1B and C). Equation 1 & 2 described the distortion that was fitted to a plane in x and y, independently, where k is the plane's slopes. R2=0.9404 for the x-axis fit and R2=0.9546 for the y-axis fit. The fitted polynomial functions were used to determine chromatic aberration in any position in the FOV.

dx(x,y)=kxxx+kxyy Equation 1

dy(x,y)=kyxx+kyyy Equation 2

As a result of knowing centroid positions in x and y with high precision at multiple locations in the two channels we were able to generate a unique vector map that characterized the chromatic aberration of the specific objective that we used relative to the image detector. Because the coordinates of the vector map were relative to the position of the camera, it was important to secure the camera's position to prevent rotational movement of the detector. Interestingly, it was also found that this function could be different for every type of objective, even of the same model and, therefore, this calibration needs to be applied to every objective lens in order to obtain super-registration (see Materials and Methods: ‘Objective testing’ section). The objective's chromatic aberration between Cy5 and Cy3 was compensated for using an affine transformation resulting in a mean registration error of 7.86 nm±0.21 nm (for the entire FOV) (FIG. 1). The main contribution to having a mean registration error that is>0 is uncertainty in the centroid localization (due to signal-to-noise ratio (SNR)) detected by FISH_QUANT (see Materials and Methods: ‘Single-molecule localization’ section). The mean registration error was 65.46 nm±1.07 nm (for the entire FOV) without chromatic aberration correction.

In addition to chromatic aberration, there are other influences that cause two colors to diverge from each other, such as room environmental conditions, vibration of the apparatus by motorized components that change the position of filter sets and the z-position of the objective or specimen during z-stack acquisition, having the plane of coverslip not parallel to plane of the microscope slide, mismatches in the refractive index, cross talk, post-acquisition image analysis that performs single-molecule localization, etc. It is not possible to completely correct for all these factors. However, mechanical instability was compensated for during imaging acquisition. Each day, at least 3 fields of sub-diffraction limit sized fluorescent beads were imaged both before and after smFISH-IF images were acquired (FIG. 7). After chromatic aberration correction was applied, the mean displacement vector was calculated between the centroid positions in x and y in the two channels and v was estimated, the average linear offset in x and y-axes caused by mechanical shift (Equation 3 & 4).

dx(x,y)=(kxxx+kxyy)+vx Equation 3

dy(x,y)=(kyxx+kyyy)+vy Equation 4

Thus, the displacements dx and dy (Equation 3 & 4) were used to compensate for both chromatic aberration (described in Equation 1 & 2) and mechanical shift between Cy5 and Cy3 channels using an affine transformation. To evaluate the mechanical stability of the system an experiment was performed in which we applied the procedure that corrects for chromatic aberration and mechanical shifts, but rather than imaging smFISH-IF, diffraction limited sized beads (25 fields, n=2,300 beads) were imaged. While without any correction the mean registration error was 65.21 nm±0.61 nm (for the entire FOV), the mean error was 43.54 nm±0.39 nm (for the entire FOV) when only chromatic aberration was corrected and 20.45 nm±0.22 nm (for the entire FOV) when both chromatic aberration and mechanical shift corrections were applied (FIG. 7). These results show that both chromatic aberration and mechanical shift need to be corrected and also suggest that the optical path alignment set up and room environment were stable during imaging acquisition. Importantly, this approach does not require fiduciary markers (beads) within the biological sample field and thus, avoid introducing noise that interferes with the accurate detection of single molecules. It is worth to mention that this method corrects for chromatic aberration on the optical system and not inside the cell and only applies to fixed samples using homogenous refractive index. Therefore, this super-registration method improves the confidence with which it can be determined that two labeled objects are “colocalized” at molecular resolution.

Measurement of Intermolecular Distances and Determining the Significance of Association.

After the centroid positions in x and y were corrected for chromatic aberration and mechanical shifts in the Cy5 channel and Cy3 channel (as described above in ‘Super-registration’ section), a nearest-neighbor algorithm was first used to pair molecules between the two channels (pairing). This pairing procedure had the assumptions that no molecule could be a member of more that one pair at a time and that some molecules may remain unpaired. Then, the Euclidean distance was measured between the centroid positions in each pair. Finally, to ensure that the molecules in a pair did not occur by chance, the likelihood of finding the two molecules in close proximity was measured. Starting with the assumption that the smaller the distance between the molecules in the pair and the further away the pair was from its molecular neighbors, the more significance one can assign to the likelihood that that pair of molecules were associated. Conventionally, one would perform a Monte-Carlo simulation in which all the molecules were randomly positioned repeatedly within the region of interest (ROI) resulting in a distribution of simulated intermolecular distances. This approach uses the global molecular population within an ROI, which overestimates the significance of the interactions by homogenizing the local context of molecular densities. For this reason, a method was developed to determine the probability of molecular association for each molecular pair based on the intermolecular distances observed and the proximal context within the cell (i.e., where the RNA-protein interactions are taking place).

The significance of an association from the perspective of the channel in which the molecules were less abundant was measured (i.e., mRNA (red spots), FIG. 2D). However, it is also possible to make the observation from either channel or to even combine the results as a sum of squares. Once an intermolecular pair is identified, the geometric boundary for the analysis was the distance from the ‘red’ molecule of the pair to the next closest ‘red’ molecule (FIG. 2D, outer dashed line). A 10,000 iteration Monte-Carlo simulation was then performed using the number of molecules counted from both channels ('red' and ‘green’) within that area. A distribution of 10,000 distances (one for every iteration) was obtained, each of which was the smallest intermolecular distance measured among the randomly positioned molecules. Then calculated was the percentile rank as the ratio of the number of times that the simulation yielded a distance that was less than or equal to the observed distance and the total number of simulations. This percentile rank expressed the probability of chance association with values that ranged from 0 to 1. The probability of chance association described the likelihood that two molecules in a pair would have been the same distance apart (or closer) than observed distance if randomly positioned given the local molecular density in each channel. The lower the probability of chance association, the more significant the association. The significance of association was measured using the molecular density immediately adjacent to the association observed by limiting the area where the simulation was performed. Also measured was the significance of association in cell body of neurons where the molecular density is higher than in dendrites. The ratio of association for MCP-MBS in cell body and in dendrites were found comparable (87% in cell body Vs. 85% in dendrites). This suggests that the statistical analysis is capable of determining molecular interactions in crowding areas of the cell with similar significant association. Thus, the results were independent of the ROI and global spatial molecular distribution, either by number or by density, in either channel yielding a more robust evaluation of the molecular associations' significance. It is important to note that the statistical analysis breaks down when there is so much crowding that the optimal distance (OD) cannot distinguish between associations by chance from meaningful associations. Since the signal-to-noise of the OD is about 6:1 (FIG. 2E), the concentration of one component would need to be 6 times higher. This is a concentration not seen, and molecular crowding would require that the space would be almost entirely filled with a single molecule, not a biologically relevant situation. If this were the case, we could compensate by reducing the OD to less than 69 nm, thereby reducing the effective amount of the higher component.

Registration as a Molecular Ruler.

The precision of the registration measured for the antibodies to the MCP-GFP and the probes to the MBS suggests that they should be able to detect when fluorescent signals from smFISH were positioned at different physical distances along the mRNA. To test whether these intermolecular distances could be measured accurately, β-actin mRNA was imaged by using specific RNA FISH probes to the ORF (at 500 nts average distance from the MBS) and anti-GFP antibodies to the MCP-GFP (FIG. 9A). It was found that the ratio of association at the OD (=69 nm) between β-actin mRNA and MCP-GFP decreased to 65% compared to 85% for MBS probes (FIG. 9B). The interaction distances were shifted to higher values suggesting a longer OD (85 nm compared to 69 nm using the MBS FISH probes) (FIG. 9C-9E). After the new OD of 85 nm was taken into account, the association recovered to 80% (FIG. 9B). These results demonstrate that physical distances between the locations where two fluorophores are positioned could be mapped with 10-20 nm precision. This has implications for evaluating the distance between any two pairs of fluors, depending on their molecular separation and it could be used in this work to standardize the evaluation of real RNA-protein interactions regardless of where they may bind on the mRNA.

Mouse Embryonic Fibroblast Cell Culture and Cell Lysis Procedure.

Mouse embryonic fibroblasts (MEFs) were isolated from E14 embryos and immortalized with SV40 large T antigen as previously described in (18), and maintained in 10-cm culture dishes with DMEM medium (Invitrogen) containing 10% heat-inactivated FBS (Sigma) and 1% penicillin and streptomycin (Invitrogen) at 37° C. and 5% CO₂.

For pull down experiments, 300 million cells were grown in 15-cm dish (approx. 50 dishes per condition). Healthy and not density-arrested cell cultures (70-80% confluence) were rinsed twice with ice-cold 1×PBS and collected in 2-mL of ice-cold 1×PBS containing 1 mM PMSF per dish using a cell scraper, transferred into ice-cold 15-mL sterile polypropylene centrifuge tube, centrifuged at 1000 RPM (300 RCF) for 10 minutes at 4° C. Then, the cell pellet was washed once with 10-ml ice-cold 1×PBS containing 1 mM PMSF, flash-frozen in liquid nitrogen, and stored at −80° C. until cell lysis.

For cell lysis, cell pellets were thawed upon the addition of 3 volumes of PCV (Packed Cell Volume) of ice-cold complete lysis buffer (50 mM Tris-HCL pH 7.4, 100 mM NaCl, 1 mM MgCl2, 0.1 mM CaCl2, 1% NP-40, 0.5% DOC, 0.1% SDS supplemented with 1 mM PMSF, 1 mM DTT, Protease Inhibitor cocktail (Roche), 100 U/ml RNaseOUT (Invitrogen)), incubated for 10 minutes on ice (swelling) and frozen/thawed twice in liquid nitrogen. Cell debris was pelleted by centrifugation at maximum speed for 10 minutes at 4° C. and the supernatant removed and transferred to a new ice-cold tube. Total protein concentration was determined by using Coomassie Plus (Bradford) Assay Reagent (Thermo Scientific).

Construction of the PP7-Tagged β-Actin 3′-UTR RNA.

A fragment containing the last 60 nucleotides of the ORF and the first ninety nucleotides of the 3′-UTR of β-actin mRNA was amplified by PCR from the pcDNA3-b-actin-3′UTR plasmid by using the following primers: T7_actbFwd: 5′-CTAATACGACTCACTATAGGGGCAAGCAGGAGTACGATGAGTCC-3′ (SEQ ID NO:1); actb_3UTR_pp7_1Rev(actbpp7R): 5′-taGGAGCGACGCCATATCGTCTGCTCCtataGCCATGCCAATGTTGTCTC-3′ (SEQ ID NO:2); T7_actb_middleFwd: 5′-CTAATACGACTCACTATAGGGCGGTGAAGGCGACAGCAGTTGG-3′ (SEQ ID NO:3). Control RNA was prepared from pLacZ plasmid by using the following primers: T7_LacZFwd: 5′-CTAATACGACTCACTATAGGGCAGCCCTTCCCGGCTGTGCCG-3′ (SEQ ID NO:4) and LacZpp7Rev: 5′-taGGAGCGACGCCATATCGTCTGCTCCtataATCAGCGACTGATCCACCCAGTCC-3′ (SEQ ID NO:5).

T7 promoter (bold) and PP7 stem-loop (underlined) sequence were added into the forward and reverse primers, respectively. The PCR product obtained was then in vitro transcribed by using MEGAshortscript T7 transcription kit (Ambion) following manufactures' instructions.

PP7-MBP recombinant protein purification. PP7 coat protein (PCP) were cloned by PCR into a derivative of pMalc vector (New England BioLabs) that contains a Tobacco Etch virus (TEV) protease site after the Maltose-Binding Protein (MBP). A C-terminal 6×His tag was added by PCR to ensure purification of the intact fusion protein as was described previously by (14). The vector was transformed into Escherichia coli strain Rosetta2 (EMD Biosciences) and recombinant protein was induced with 1 mM IPTG for 4 h at 37° C. Cell pellets were resuspended in lysis buffer (50 mM Tris at pH 7.5, 1.5 M NaCl, 1 mM EDTA, 1 mM DTT) supplemented with one Complete EDTA-free protease inhibitor tablet (Roche), and were lysed by sonication. Cell debris was removed by centrifugation, and the soluble fusion protein was purified by amylose affinity chromatography (New England BioLabs) followed by either TALON affinity (Clontech) or anion exchange (GE Healthcare) chromatography.

Staining of Gels, Mass Spectrometry (MS) and Western Blot Analysis.

Following SDS-PAGE, protein gels were stained either by silver staining (SilverQuest™ Staining Kit, Invitrogen) or by a fast and sensitive Coomassie-dye (GelCode™ Blue Safe Protein Stain (Thermo Scientific)). Gel lanes were excised in slices and analyzed by tandem Liquid Chromatography-Mass Spectrometry (LC-MS/MS) at the Proteomic Resource Center at The Rockefeller University. In parallel, candidate proteins were identified by Western Blot analysis. Ten μl of eluates were separated in SDS-PAGE and transferred to nitrocellulose membranes (Life Technologies). After blocking in 1% milk in 1×PBS-Tween, membranes were incubated with primary antibody in blocking solution before they were washed and incubated with infrared-labeled secondary antibodies for 40 minutes at room temperature. Signal was detected by using Odyssey, Infrared Imaging System (LI-COR, Biosciences).

Gene Ontology Analysis.

IPA Knowledge Base 9 (Ingenuity Systems; http://www.ingenuity.com/products/ipa) was used to investigate the functional relationship among the proteins identified by the RNA affinity purification procedure. The enrichment of GO terms of selected genes to molecular and cellular function categories was determined. The p-value, based on a right-tailed Fisher's exact test, considered the number of identified genes and the total number of molecules known to be associated with these categories in the IPA Knowledge Base. Only statistically significantly enriched GO terms with p-value less than 0.05 were considered.

RNA Immunoprecipitation (RIP).

Cells were scraped, rinsed with ice-cold 1×PBS and lysed in ice-cold 10 mM HEPES-KOH pH 7.0, 100 mM KCl, 5 mM MgCl₂, 0.5% NP-40 supplemented by 1 mM PMSF, 1 mM DTT, Protease Inhibitor cocktail (Roche) and 100 U/ml RNAseOUT (Invitrogen). Cell lysates were mixed with 50-μl Dynabeads-protein A (Invitrogen) and pre-cleared for 1 hour at 4° C. (to reduce background). In parallel, pre-washed Dynabeads-protein A (50 μl/per reaction tube) resuspended in NT2 buffer (50 mM Tris-HCl pH 7.4, 150 mM NaCl, 1 mM MgCl₂, 0.05% NP-40) supplemented with 1 mM PMSF and 100 U/ml RNAseOUT (Invitrogen) were incubated with either anti-Ig (nonspecific control) or specific antibodies with gentle rotation for 1:30 hour at 4° C. Subsequently, magnetic beads were washed five times with NT2 buffer (1-ml) and incubated with pre-cleared cell lysate supplemented with 200 U RNAseOUT, 1 mM DTT and 20 mM EDTA pH 8.0 in NT2 buffer for 3 hours at 4° C. tumbling end over end. Magnetic beads were then washed five times with ice-cold NT2 buffer and then resuspended in 100 μl NT2 buffer supplemented with 0.1% SDS and 30 μg Proteinase K (Invitrogen) for 30 minutes at 55° C., flicking the tube occasionally. RNA was then extracted by adding phenol:chloroform:isoamyalcohol (25:24:1) (Sigma) and precipitated overnight at −20° C. with 2-propanol supplemented with 300 mM Sodium Acetate pH 5.2 and 1 μl glycogen (Roche) as a carrier. After centrifugation at 20,000 RCF for 20 minutes at 4° C., RNA pellet was air-dried and resuspened in RNAse-free water and subsequently treated with DNAse-TURBO following manufacture specifications (Ambion). The amount of RNA was then quantified using NanoDrop (Thermo Fisher Scientific) and cDNAs were synthesized using SuperScript III First-Strand Synthesis System for RT-PCR□ (Invitrogen). Equal amounts of cDNA were subjected to semi-quantitative PCR using Platinum Taq polymerase (Invitrogen) using the following specific pair of primers to detect β-actin and gapdh mRNA, as was described in (18): Actb_MBS(2009-29)Fwd: 5′-GATCTGCGCGCGATCGATATCAGCGC-3′ (SEQ ID NO:5); Actb_MBS(2009-30)Rev: 5′-GCCAGCCCTGGCTGCCTCAACACCTC-3′ (SEQ ID NO:6); GAPDH(2009-15)Fwd: 5′-GAGCGAGACCCCACTAACATCAAATG-3′ (SEQ ID NO:7); GAPDH(2009-16)Rev: 5′-CAGGATGCATTGCTGACAATCTTGAG-3′ (SEQ ID NO:8).

Plasmids and Lentivirus Generation.

Coding sequences for tandem-dimer MCP-GFP (tdMCP-mEos2-GFP) and GFP-ZBP1 were cloned into the lentivirus expression vector. Lentivirus particles were produced as follows: plasmids for ENV (pMD2.VSVG), packaging (pMDLg/pRRE), REV (pRSV-Rev) and the expression vector (gift from A. Follenzi) were mixed and transfected into HEK 293T cells using Lipofectamine 2000 reagent (Invitrogen) as per manufacturer's instructions. Expression of the insert was under the control of the UbC promoter. The virus-containing supernatant was harvested and concentrated using Lenti-X concentrator (Clontech) as per manufacturer's instructions. The viral particles were resupended in Neurobasal A and stored at −80° C. for subsequent infection of neurons in culture. DNA constructs used in this work are available at Addgene.

RNA FISH Probes.

MBS probes (Invitrogen) were used to detect MBS cassette present in β-actin mRNA 3′-UTR in MBS cells as was described in (18). Each probe was labeled at both ends with Cy3 fluorescent dye (GE Healthcare). β-actin ORF probes (Invitorgen) were used to detect β-actin mRNA as was described in (18). Each probe was labeled at both ends with Cy3 fluorescent dye (GE Healthcare). CaMKII probes (Stellaris RNA FISH probes, Biosearch Technologies) were used to detect CaMKII mRNA. Each probe was labeled at the 5′-end with Quasar570 fluorescent dye. Spino probes (Stellaris RNA FISH probes, Biosearch Technologies) were used to detect spinophilin mRNA. Each probe was labeled at the 5′-end with Quasar570 fluorescent dye.

Antibodies.

For Western Blot, polyclonal rabbit anti-zbp1 (gift from Stefan Hüttelmaier), rabbit polyclonal anti-hnRNPA/B (M-36) (Santa Cruz (sc-98810), rabbit polyclonal anti-YB1 (Abcam (ab12148), rabbit monoclonal anti-KHDRBS1/SAM68 (Lifespan Biosciences (EPR3232), rabbit polyclonal anti-Sam68 (C-20) (Santa Cruz (sc-333), gift from Mat Klein), rabbit polyclonal anti-RNA Helicase A (Dhx9) (Abcam (ab26271), rabbit polyclonal anti-FMRP (Abcam (ab17722), mouse monoclonal anti-tubulin-alpha (DMA1) (Sigma (T6199), mouse monoclonal anti-MBP (New England BioLabs (E8032S), mouse monoclonal anti-beta-actin clone AC15 (sigma (A1978)), rabbit polyclonal anti-IGFBP2/IMP2 (MBL (RN008P), rabbit polyclonal anti-MRCL3/MRLC2/MYL9 (FL-172) (Santa Cruz (sc-15370), anti-rabbit and anti-mouse IgG (H&L) (goat) antibodies IRDye680 and IRDye800 conjugated (Rockland). For IF, antibodies used were: chicken anti-GFP (1:5000; Ayes Labs, Inc. (GFP-1010)), rabbit polyclonal anti-MAP2 (Millipore (AB5622); dilution 1/2500), mouse monoclonal anti-MAP2 (Sigma (M4403); dilution 1/1500), rabbit anti-anti-hnRNPA/B (M-36) (Santa Cruz (sc-98810), anti-YBOX1 ((Abcam (ab12148), anti-sam68 (Lifespan Biosciences (EPR3232), rabbit polyclonal anti-Sam68 (C-20) (Santa Cruz (sc-333)), anti-Dhx9 (Abcam (ab26271), anti-FMRP (Abcam (ab17722), mouse monoclonal anti-hnRNPU (Sigma (R6278)), gift from Stefan Hüttelmaier), mouse monoclonal anti-hnRNPE2 (PCBP2) (Abnova (H00005094), gift from Stefan Hüttelmaier), mouse monoclonal IGF2BP1/IMP1 (MBL (RN001M)), polyclonal rabbit anti-zbp1 (gift from Stefan Hüttelmaier. Alexa Fluor labeled anti-chicken, -mouse and -rabbit secondary antibodies were used (Life Technologies; dilution 1/1000).

### REFERENCES

1. Eliscovich C, Buxbaum A R, Katz Z B, & Singer R H (2013) mRNA on the move: the road to its biological destiny. The Journal of biological chemistry 288(28):20361-20368.
2. Glisovic T, Bachorik J L, Yong J, & Dreyfuss G (2008) RNA-binding proteins and post-transcriptional gene regulation. FEBS letters 582(14):1977-1986.
3. Kao D I, Aldridge G M, Weiler I J, & Greenough W T (2010) Altered mRNA transport, docking, and protein translation in neurons lacking fragile X mental retardation protein. Proceedings of the National Academy of Sciences of the United States of America 107(35):15601-15606.
4. King O D, Gitler A D, & Shorter J (2012) The tip of the iceberg: RNA-binding proteins with prion-like domains in neurodegenerative disease. Brain research 1462:61-80.
5. Lagier-Tourenne C, Polymenidou M, & Cleveland D W (2010) TDP-43 and FUS/TLS: emerging roles in RNA processing and neurodegeneration. Hum Mol Genet 19(R1):R46-64.
6. Liu-Yesucevitz L, et al. (2011) Local RNA translation at the synapse and in disease. The Journal of neuroscience: the official journal of the Society for Neuroscience 31(45):16086-16093.
7. Lukong K E, Chang K W, Khandjian E W, & Richard S (2008) RNA-binding proteins in human genetic disease. Trends Genet 24(8):416-425.
8. Nussbacher J K, Batra R, Lagier-Tourenne C, & Yeo G W (2015) RNA-binding proteins in neurodegeneration: Seq and you shall receive. Trends Neurosci 38(4):226-236.
9. Paronetto M P, Achsel T, Massiello A, Chalfant C E, & Sette C (2007) The RNA-binding protein Sam68 modulates the alternative splicing of Bcl-x. The Journal of cell biology 176(7):929-939.
10. Cook K B, Hughes T R, & Morris Q D (2015) High-throughput characterization of protein-RNA interactions. Briefings in functional genomics 14(1):74-89.
11. Oeffinger M (2012) Two steps forward—one step back: advances in affinity purification mass spectrometry of macromolecular complexes. Proteomics 12(10):1591-1608.
12. Mili S & Steitz J A (2004) Evidence for reassociation of RNA-binding proteins after cell lysis: implications for the interpretation of immunoprecipitation analyses. Rna 10(11):1692-1694.
13. Riley K J & Steitz J A (2013) The “Observer Effect” in genome-wide surveys of protein-RNA interactions. Molecular cell 49(4):601-604.
14. Chao J A, et al. (2010) ZBP1 recognition of beta-actin zipcode induces RNA looping. Genes & development 24(2):148-158.
15. Huttelmaier S, et al. (2005) Spatial regulation of beta-actin translation by Src-dependent phosphorylation of ZBP1. Nature 438(7067):512-515.
16. Patel V L, et al. (2012) Spatial arrangement of an RNA zipcode identifies mRNAs under post-transcriptional control. Genes & development 26(1):43-53.
17. Ross A F, Oleynikov Y, Kislauskis E H, Taneja K L, & Singer R H (1997) Characterization of a beta-actin mRNA zipcode-binding protein. Molecular and cellular biology 17(4):2158-2165.
18. Lionnet T, et al. (2011) A transgenic mouse for in vivo detection of endogenous labeled mRNA. Nature methods 8(2):165-170.
19. Bertrand E, et al. (1998) Localization of ASH1 mRNA particles in living yeast. Molecular cell 2(4):437-445.
20. Mueller F, et al. (2013) FISH-quant: automatic counting of transcripts in 3D FISH images. Nature methods 10(4):277-278.
21. Wu B, Chao J A, & Singer R H (2012) Fluorescence fluctuation spectroscopy enables quantitative imaging of single mRNAs in living cells. Biophysical journal 102(12):2936-2944.
22. Chao J A, Patskovsky Y, Almo S C, & Singer R H (2008) Structural basis for the coevolution of a viral RNA-protein complex. Nature structural & molecular biology 15(1):103-105.
23. Sinnamon J R, Waddell C B, Nik S, Chen E I, & Czaplinski K (2012) Hnrpab regulates neural development and neuron cell survival after glutamate stimulation. Rna 18(4):704-719.
24. Klein M E, Younts T J, Castillo P E, & Jordan B A (2013) RNA-binding protein Sam68 controls synapse number and local beta-actin mRNA metabolism in dendrites. Proceedings of the National Academy of Sciences of the United States of America 110(8):3125-3130.
25. Bassell G J & Warren S T (2008) Fragile X syndrome: loss of local mRNA regulation alters synaptic development and function. Neuron 60(2):201-214.
26. Lagache T, Sauvonnet N, Danglot L, & Olivo-Marin J C (2015) Statistical analysis of molecule colocalization in bioimaging. Cytometry. Part A: the journal of the International Society for Analytical Cytology 87(6):568-579.
27. Ule J, et al. (2003) CLIP identifies Nova-regulated RNA networks in the brain. Science 302(5648): 1212-1215.
28. Hafner M, et al. (2010) Transcriptome-wide identification of RNA-binding protein and microRNA target sites by PAR-CLIP. Cell 141(1):129-141.
29. Ule J, Jensen K, Mele A, & Darnell R B (2005) CLIP: a method for identifying protein-RNA interaction sites in living cells. Methods 37(4):376-386.
30. Licatalosi D D, et al. (2008) HITS-CLIP yields genome-wide insights into brain alternative RNA processing. Nature 456(7221):464-469.
31. Zhang C & Darnell R B (2011) Mapping in vivo protein-RNA interactions at single-nucleotide resolution from HITS-CLIP data. Nature biotechnology 29(7):607-614.
32. Konig J, et al. (2010) iCLIP reveals the function of hnRNP particles in splicing at individual nucleotide resolution. Nature structural & molecular biology 17(7):909-915.
33. Friedersdorf M B & Keene J D (2014) Advancing the functional utility of PAR-CLIP by quantifying background binding to mRNAs and lncRNAs. Genome biology 15(1):R2.
34. Castello A, et al. (2012) Insights into RNA biology from an atlas of mammalian mRNA-binding proteins. Cell 149(6):1393-1406.
35. Jonson L, et al. (2007) Molecular composition of IMP1 ribonucleoprotein granules. Molecular & cellular proteomics: MCP 6(5):798-811.
36. Weidensdorfer D, et al. (2009) Control of c-myc mRNA stability by IGF2BP1-associated cytoplasmic RNPs. Rna 15(1):104-115.
37. Lin Q, Taylor S J, & Shalloway D (1997) Specificity and determinants of Sam68 RNA binding. Implications for the biological function of K homology domains. The Journal of biological chemistry 272(43):27274-27280.
38. Itoh M, Haga I, Li Q H, & Fujisawa J (2002) Identification of cellular mRNA targets for RNA-binding protein Sam68. Nucleic acids research 30(24):5452-5464.
39. Snee M, Kidd G J, Munro T P, & Smith R (2002) RNA trafficking and stabilization elements associate with multiple brain proteins. Journal of cell science 115 (Pt 23):4661-4669.
40. Raju C S, et al. (2011) In neurons, activity-dependent association of dendritically transported mRNA transcripts with the transacting factor CBF-A is mediated by A2RE/RTS elements. Mol Biol Cell 22(11):1864-1877.
41. Raju C S, et al. (2008) In cultured oligodendrocytes the AB-type hnRNP CBF-A accompanies MBP mRNA bound to mRNA trafficking sequences. Mol Biol Cell 19(7):3008-3019.
42. Fukuda N, et al. (2013) The transacting factor CBF-A/Hnrnpab binds to the A2RE/RTS element of protamine 2 mRNA and contributes to its translational regulation during mouse spermatogenesis. PLoS Genet 9(10):e1003858.
43. Wu B, Buxbaum A R, Katz Z B, Yoon Y J, & Singer R H (2015) Quantifying Protein-mRNA Interactions in Single Live Cells. Cell 162(1):211-220.
44. Xu D, Jaroszewski L, Li Z, & Godzik A (2014) AIDA: ab initio domain assembly server. Nucleic acids research 42(Web Server issue):W308-313.

1. A method of improving the performance of a fluorescence microscopy imaging system comprising an optical objective lens, a field of view, an imaging detector, and at least a first and a second fluorescent molecule, each of which fluoresces at a different wavelength than the other and each of which has a different excitation radiation peak than the other fluorescent molecule, the method comprising:
providing in a field of view of the fluorescence microscopy system a plurality of fluorescent beads capable of fluorescing at each of the different wavelengths of the first and second fluorescent molecules, wherein the beads have a diameter lower than a diffraction limit of the optical fluorescence microscopy system;
irradiating the plurality of fluorescent beads at an excitation radiation peak of the first fluorescent molecule and sequentially imaging the fluorescence of each of the plurality of beads within field of view of the fluorescence microscopy system and at a plurality of different z-dimension positions;
irradiating the plurality of fluorescent beads at an excitation radiation peak of the second fluorescent molecule and sequentially imaging the fluorescence of each of the plurality of beads within field of view of the fluorescence microscopy system and at a plurality of different z-dimension positions;
locating, from a point spread function of the fluorescence of each bead imaged at the excitation radiation peak of the first fluorescent molecule, the x,y coordinates of a centroid for each bead at each z-dimension position;
locating, from a point spread function of the fluorescence of each bead imaged at the excitation radiation peak of the second fluorescent molecule, the x,y coordinates of a centroid for each bead at each z-dimension position;
calculating, from a difference in the centroid x,y coordinates for each bead at the first and second excitation radiation peaks, a displacement vector for each x,y coordinate in the field of view at each z-dimension position, so as to thereby determine a displacement vector map for the optical objective of the fluorescence microscopy system;
applying the displacement vector map to imaging data obtained for the first and second fluorescent molecule so as to generate a fluorescence data image corrected for chromatic aberration in the optical objective of the fluorescence microscopy system.

2. The method of claim 1, wherein the beads are broad spectrum fluorescent beads.

3. The method of claim 1, wherein the beads are less than 250 nm in diameter

4. The method of claim 1, wherein the beads are 90-110 nm in diameter.

5. The method of claim 1, wherein the beads are 100 nm in diameter

6. The method of claim 1, wherein the optical objective's chromatic aberration between the excitation radiation peak of the first and second fluorescent molecule is corrected for by applying an affine transformation.

7. The method of claim 1, wherein the displacement vector map applied to imaging data obtained for the first and second fluorescent molecule so as to generate a fluorescence data image corrected for chromatic aberration is applied as an affine transformation matrix.

8. A method of correcting for chromatic aberration in a fluorescence microscopy system comprising an optical objective lens, a field of view, an imaging detector, and at least a first and a second fluorescent molecule, each of which fluoresces at a different wavelength than the other and each of which has a different excitation radiation peak than the other fluorescent molecule, the method comprising:
providing in a field of view of the fluorescence microscopy system a plurality of fluorescent beads capable of fluorescing at each of the different wavelengths of the first and second fluorescent molecules, wherein the beads have a diameter lower than a diffraction limit of the optical fluorescence microscopy system;
irradiating the plurality of fluorescent beads at an excitation radiation peak of the first fluorescent molecule and sequentially imaging the fluorescence of each of the plurality of beads within field of view of the fluorescence microscopy system and at a plurality of different z-dimension positions;
irradiating the plurality of fluorescent beads at an excitation radiation peak of the second fluorescent molecule and sequentially imaging the fluorescence of each of the plurality of beads within field of view of the fluorescence microscopy system and at a plurality of different z-dimension positions;
locating, from a point spread function of the fluorescence of each bead imaged at the excitation radiation peak of the first fluorescent molecule, the x,y coordinates of a centroid for each bead at each z-dimension position;
locating, from a point spread function of the fluorescence of each bead imaged at the excitation radiation peak of the second fluorescent molecule, the x,y coordinates of a centroid for each bead at each z-dimension position;
calculating, from a difference in the centroid x,y coordinates for each bead at the first and second excitation radiation peaks, a displacement vector for each x,y coordinate in the field of view at each z-dimension position, so as to thereby determine a displacement vector map for the optical objective of the fluorescence microscopy system;
applying the displacement vector map to imaging data obtained for the first and second fluorescent molecule so as to generate a fluorescence data image corrected for chromatic aberration.

9. A kit comprising a plurality of broad spectrum fluorescent beads and a non-transitory computer readable medium having instructions thereon for performing the method of claim 1 in a fluorescence microscopy imaging system.

10. A method of detecting at least two co-localized fluorescent markers, wherein each of the two markers has a different emission spectrum, in a field of view of a fluorescence microscopy imaging system, the method comprising
subjecting an in vitro or in vivo system which has been preloaded with the two markers, wherein at least a portion of the in vitro or in vivo system is within the field of view of the fluorescence microscopy imaging system to irradiation at an excitation spectrum peak of each of the two different markers;
obtaining a fluorescence image for each two markers, when subjected to irradiation, with an optical objective of the fluorescence microscopy imaging system;
correcting the fluorescence images obtained for chromatic aberration of the optical objective at each of the different emission spectrums of the two fluorescent markers by the method of claim 8;
determining if the chromatic aberration-corrected fluorescence images show two colocalized different fluorescent markers, so as to thereby detect at least two co-localized fluorescent markers.

11. The method of claim 10, wherein each fluorescent marker is bound to a separate biological molecule.

12. The method of claim 11, wherein the intermolecular distance for each of the two bound molecules is calculated from adjacent chromatic aberration-corrected fluorescent dye positions.

13. A non-transitory computer-readable medium coupled to the one or more data processing apparatus coupled to an optical microscope fluorescence imaging system, the medium having instructions stored thereon which, when executed by the one or more data processing apparatus, cause the one or more data processing apparatus to perform a method of claim 1.

14. Also provided is a system for improving the performance of a fluorescence microscopy imaging system, comprising:
one or more data processing apparatus;
a graphical user interface; and
a non-transitory computer-readable medium coupled to the one or more data processing apparatus having instructions stored thereon which, when executed by the one or more data processing apparatus, and coupled to an optical microscope fluorescence imaging system, cause the one or more data processing apparatus to perform a method of claim 1.


SEQUENCE LISTING (9 sequences)

SEQ ID NO: 1 - DNA, 44 bp, Artificial Sequence
  primer T7_actbFwd directed to mouse sequence
ctaatacgactcactataggggcaagcaggagtacgatgagtcc

SEQ ID NO: 2 - DNA, 50 bp, Artificial Sequence
  primer actb_3UTR_pp7_1Rev(actbpp7R) directed to mouse sequence
taggagcgacgccatatcgtctgctcctatagccatgccaatgttgtctc

SEQ ID NO: 3 - DNA, 43 bp, Artificial Sequence
  primer T7_actb_middleFwd directed to mouse sequence
ctaatacgactcactatagggcggtgaaggcgacagcagttgg

SEQ ID NO: 4 - DNA, 41 bp, Artificial Sequence
  primer T7_LacZFwd directed to mouse sequence
ctaatacgactcactatagggcagcccttcccggctgtgcc

SEQ ID NO: 5 - DNA, 55 bp, Artificial Sequence
  Primer LacZpp7Rev directed to mouse sequence
taggagcgacgccatatcgtctgctcctataatcagcgactgatccacccagtcc

SEQ ID NO: 6 - DNA, 26 bp, Artificial Sequence
  primer Actb_MBS(2009-30)Rev directed to mouse sequence
gccagccctggctgcctcaacacctc

SEQ ID NO: 7 - DNA, 26 bp, Artificial Sequence
  primer GAPDH(2009-15)Fwd directed to mouse sequence
gagcgagaccccactaacatcaaatg

SEQ ID NO: 8 - DNA, 26 bp, Artificial Sequence
  primer GAPDH(2009-16)Rev directed to mouse sequence
caggatgcattgctgacaatcttgag

SEQ ID NO: 9 - DNA, 26 bp, Artificial Sequence
  primer Actb_MBS(2009-29)Fwd directed to mouse sequence
gatctgcgcgcgatcgatatcagcgc